(19) United States
(12) Patent Application Publication
(10) Pub. No.: US 2022/0054189 A1
(43) Pub. Date: Feb. 24, 2022

(54) UNIFORM DIFFUSION OF RADIOFREQUENCY HEATING BY ELECTRODE ARRAY

(71) Applicant: Aesthetics Biomedical, Inc., Phoenix, AZ (US)

(72) Inventor: Shaun Wootten, Tempe, AZ (US)

(21) Appl. No.: 17/516,422

(22) Filed: Nov. 1, 2021

Related U.S. Application Data
(63) Continuation of application No. PCT/US2020/030827, filed on Apr. 30, 2020.
(60) Provisional application No. 62/842,384, filed on May 2, 2019.

(51) Int. Cl. A61B 18/14

(52) U.S. Cl. CPC A61B 18/14; A61B 2018/00589; A61B 2018/143

(57) ABSTRACT

A microneedling system may reciprocate a plurality of microneedles disposed on a handpiece into the skin of a patient. The handpiece may have a plurality of positive and negative electrodes in the form of microneedles or surface electrodes arranged across an array. The microneedles and/or electrode plates may deliver RF energy to the patient for inducing collagen coagulation and regeneration. The electrodes may be arranged such that each electrode is positioned adjacent a closest electrode of opposite polarity. There may be an uneven number of positive and negative electrodes. Central electrodes may be surrounded by at least three adjacent closest electrodes of opposite polarity. The electrodes may be arranged in a hexagonal or other polygonal manner. The electrodes may be arranged to provide uniform distribution of energy, heating, and effectively to some extent damage the entire discrete area that encloses the positive and negative electrodes.

### FIELD OF THE DISCLOSURE

The disclosure herein relates to skin treatment devices for inducing collagen regeneration.

### BACKGROUND

Collagen is the most common and abundant form of protein in the body. It is found in many tissues of the muscles, bones, tendons, blood vessels, the digestive system, and skin. As people age, their bodies produce less collagen. This lack of collagen results in the common signs of aging. Wrinkles, sagging skin that has lost its elasticity, and stiff joints are all signs that the body is producing less collagen. When collagen levels are high, the skin is soft, smooth, and firm. Collagen helps the skin cells renew and repair themselves.

One approach to skin rejuvenation is to physically inject collagen into the skin. This gives an appearance of fullness or plumpness and the offending lines are smoothened. Bovine collagen has been used for this purpose. Unfortunately, this is not a long-lasting or complete fix for the problem and there are frequent reports of allergic reactions to the collagen injections.

To overcome some of the issues associated with the invasive procedures, laser and radio frequency energy based wrinkle reduction treatments have been proposed. For example, U.S. Pat. No. 6,277,116 (the '116 patent) discloses a device that delivers RF energy in an attempt to achieve uniform heating at a prescribed depth in the dermis layer using a conductive fluid. Others for example U.S. Pat. No. 9,095,357 (the '357 patent), describe devices to regulate delivery of RF energy to induces pattern of fractional damage by the RF energy in the dermal layer. The '357 patent describes adjacent regions of thermal damage next to undamaged regions. It is believe that fractional technology is safer because the damage occurs within smaller subvolumes or islets within the larger volume being treated. The tissue surrounding the islets is spared from the damage. Because the resulting islets are surrounding by neighboring healthy tissue the healing process is thorough and fast. However, fractional or patterned heat generation often forms an undesired and uneven result. Thus, there is a need for an improved device that can provide uniform heating with desired and even results.

### SUMMARY

Disclosed herein are devices configured for providing a therapeutic treatment to a patient (e.g., a human patient). The therapeutic treatment can be a cosmetic treatment, such as collagen induction therapy.

In one aspect, a device is provided for delivering electrical energy to the skin of a patient, the device comprising: an array comprising a plurality of electrodes arranged across a two-dimensional surface of the array, each electrode being configured to be placed into contact with the skin of the patient and/or to be inserted into the skin of the patient, wherein the plurality of electrodes comprises a plurality of positive electrodes and a plurality of negative electrodes, wherein each negative electrode is positioned adjacent to a positive electrode and wherein each positive electrode is positioned adjacent to a negative electrode.

In an embodiment of the first aspect, the array comprises alternating rows or columns of positive and negative electrodes.

In an embodiment of the first aspect, the plurality of electrodes comprise a plurality of central electrodes, each central electrode being uniformly surrounded by at least three adjacent closest electrodes of an opposite polarity.

In an embodiment of the first aspect, each central electrode is uniformly surrounded by at least four adjacent closest electrodes of an opposite polarity.

In an embodiment of the first aspect, the plurality of negative electrodes and the plurality of positive electrodes are arranged in a checkerboard fashion.

In an embodiment of the first aspect, each central electrode is uniformly surrounded by at least five adjacent closest electrodes of an opposite polarity.

In an embodiment of the first aspect, the plurality of electrodes are arranged in a pentagonal pattern.

In an embodiment of the first aspect, each central electrode is uniformly surrounded by at least six adjacent closest electrodes of an opposite polarity.

In an embodiment of the first aspect, the plurality of electrodes are arranged in a hexagonal pattern.

In an embodiment of the first aspect, the array forms at least one zone bordered by at least three electrodes such that neither a path between any one of the plurality of positive electrodes and a closest negative electrode nor a path between any one of the plurality of negative electrodes and a closest positive electrode crosses the zone.

In an embodiment of the first aspect, the plurality of electrodes comprises a 1:1 ratio of negative electrodes to positive electrodes.

In an embodiment of the first aspect, the plurality of electrodes comprises more than a 1:1 ratio of negative electrodes to positive electrodes.

In an embodiment of the first aspect, the plurality of electrodes comprises less than a 1:1 ratio of negative electrodes to positive electrodes.

In an embodiment of the first aspect, at least one central electrode shares a closest adjacent electrode of opposite polarity with another central electrode.

In an embodiment of the first aspect, none of the central electrodes shares a closest adjacent electrode of opposite polarity with another central electrode.

In an embodiment of the first aspect, each central electrode is a plate surface electrode.

In an embodiment of the first aspect, the plate surface electrodes are polygonal.

In an embodiment of the first aspect, the polygon has the same number of sides as the number of adjacent closest electrodes of opposite polarity.

In an embodiment of the first aspect, all of the adjacent closest electrodes of opposite polarity are microneedles.

In an embodiment of the first aspect, the surface plate electrode has a cross-sectional area at least 1.5× larger than the cross-sectional area of all of the adjacent closest electrodes of opposite polarity.

In an embodiment of the first aspect, the plurality of electrodes are distributed between a plurality of islands such that all of the electrodes inside an island are closer to an electrode of opposite polarity inside the island than an electrode of opposite polarity outside the island and such that all the electrodes outside an island are closer to an electrode of opposite polarity outside the island than an electrode of opposite polarity inside the island.

In an embodiment of the first aspect, all of the electrodes outside any one of the islands are positioned further from electrodes inside the island than any electrode inside the island is positioned relative to another electrode inside the island.

In an embodiment of the first aspect, at least one electrode outside of each of the islands is positioned as close to an electrode inside the island as the electrode inside the island is positioned to another electrode inside the island.

In an embodiment of the first aspect, each electrode from the plurality of electrodes is positioned substantially the same distance from a closest electrode of opposite polarity.

In an embodiment of the first aspect, all electrodes of a first polarity are microneedles and wherein all electrodes of a second polarity are surface electrodes.

In an embodiment of the first aspect, all of the positive electrodes are microneedles and all of the negative electrodes are surface electrodes.

In an embodiment of the first aspect, either the positive electrodes or the negative electrodes are connected to electrical ground.

In an embodiment of the first aspect, the device further comprises a handpiece, wherein the array is positioned on a distal end of the handpiece.

In an embodiment of the first aspect, the array comprises at least one microneedle configured to be inserted into the skin of the patient, and wherein the handpiece is configured to reciprocate the array such that the at least one microneedle punctures the skin of the patient.

In an embodiment of the first aspect, the device is configured to deliver the electrical energy during a period coinciding with a full insertion of the at least one microneedle into the skin of the patient.

In an embodiment of the first aspect, the device is configured to deliver the electrical energy to the skin of the patient between the plurality of positive electrodes and the plurality of negative electrodes in a waveform having frequency within the radiofrequency range.

In an embodiment of the first aspect, the device is configured to raise the temperature of the skin to a degree sufficient to induce collagen coagulation.

In an embodiment of the first aspect, the device is configured to leave a plurality of areas of skin encompassed by the array during delivery of the electrical energy, effectively untreated such that collagen is not coagulated within these areas.

In an embodiment of the first aspect, the array comprises at least one microneedle configured to penetrate the skin of the patient such that a distal tip of the needle reaches the dermis.

In a second aspect, a device is provided for delivering radio frequency energy to a dermis of a patient, the device comprising: at least one microneedle configured to penetrate the skin of the patient such that a distal tip of the microneedle reaches the dermis, wherein the microneedle comprises at least one substrate enclosed by one material, wherein the microneedle is configured to deliver a dispersion of radio frequency energy into the dermis, whereby tissue of the dermis is damaged, and wherein the microneedle is further configured to exhibit either a conducting property or a non-conducting property.

In a third aspect, a device is provided for delivering radio frequency energy to a dermis of a patient, the device comprising: at least one contact plate configured to be in contact with the skin of the patient, wherein the contact plate comprises at least one substrate enclosed by one material, wherein the contact plate is configured to deliver a dispersion of radio frequency energy into the dermis, whereby tissue of the dermis is damaged, and wherein the contact plate is further configured to exhibit either a conducting property or a non-conducting property.

In an embodiment of the second or third aspect, the device further comprises a processor configured to adjust a parameter of the radio frequency energy to select between the conducting property and the non-conducting property, wherein the parameter is at least one member selected from the group consisting of frequency, current, voltage, and conduction time.

In an embodiment of the second or third aspect, the material has a preselected dielectric relaxation value corresponding to the parameter.

In an embodiment of the second or third aspect, a thickness of the material is selected such that the conductive properties of the microneedle are preselected.

In an embodiment of the second or third aspect, the material has a preselected dielectric relaxation value corresponding to thickness of the material.

In an embodiment of the second or third aspect, the radiofrequency energy is microwave energy.

In an embodiment of the second or third aspect, damaging comprises induction of collagen coagulation.

In an embodiment of the second or third aspect, the material is an amorphous material.

In an embodiment of the second or third aspect, the material is a dielectric thin film.

In an embodiment of the second or third aspect, the material is an oxide, e.g., one or more of a silicon oxide, a zirconium oxide, a lanthanum oxide, a lanthanum zirconium oxide or a cerium oxide.

In an embodiment of the second or third aspect, the material is a polymeric material, e.g., a (substituted) paracyclophane, dichloro-di-p-xylylene, dichloro-paracyclophane, or trichloro-paracyclophane.

In an embodiment of the second or third aspect, the material is a metal oxide semiconductor.

In an embodiment of the second or third aspect, the material has a high thermal conductivity.

In an embodiment of the second or third aspect, the substrate is a metal or a metal alloy, e.g., iron, titanium, nickel, aluminum, or chromium, or a metal alloy, e.g., steel (e.g., stainless steel).

In an embodiment of the third aspect, the radio frequency energy is laser energy, and wherein the contact plate is configured to transition the laser energy from a fraction pattern to a non-fractional pattern.

In an embodiment of the third aspect, the fractional pattern is provided by an array of apertures.

In an embodiment of the third aspect, the apertures are selected from the group consisting of holes and slots

In an embodiment of the third aspect, the fractional pattern is provided by a patterned array of emitters of laser energy.

In an embodiment of the third aspect, the fractional pattern is regular.

In an embodiment of the third aspect, the fractional pattern is irregular.

Any embodiment is independently combinable, in whole or in part, with any other embodiment or aspect, in whole or in part.

### BRIEF DESCRIPTION OF THE DRAWINGS

Features of the present disclosure will become more fully apparent from the following description and appended claims, taken in conjunction with the accompanying drawings. It will be understood that these drawings depict only certain embodiments in accordance with the disclosure and, therefore, are not to be considered limiting of its scope; the disclosure will be described with additional specificity and detail through use of the accompanying drawings. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

FIG. 1 schematically depicts an example of a microneedling system having a microneedle array coupled to a housing unit.

FIG. 2 schematically depicts an image of tissue to be treated by the microneedling system having predicted volumes of coagulation from RF treatment through the microneedles at various depths and an image of the distal tip of the microneedling handpiece overlaid.

FIGS. 3A-3B schematically depict an example of an array having positive microneedle electrodes surrounded by two lateral ground surface electrode plates. FIG. 3A shows a side view of the array with the microneedles puncturing the tissue. FIG. 3B shows a bottom view of the array including the arrangement of the microneedles and surface electrodes.

FIGS. 4A-4E schematically depict examples of arrays having alternative configurations of electrodes. FIG. 4A depicts an array having alternating columns of positive and negative electrodes. FIG. 4B depicts an array having a checkerboard pattern of positive and negative electrodes. FIG. 4C depicts an array having a hexagonal pattern of negative electrodes surrounded by six positive electrodes. FIG. 4D depicts an array having a square pattern of negative electrodes surrounded by four positive electrodes. FIG. 4E depicts an array having multiple electrically isolated islands of negative electrodes surrounded by six positive electrodes.

FIGS. 5A-5D show an image processing technique for area analysis of non-uniform shapes using ImageJ. FIG. 5A is a Raw H&E image of MNRF event. FIG. 5B is a threshold color illustration to highlight electrocoagulation. FIG. 5C is a raw selection of threshold highlight for raw area analysis. FIG. 5D is a selection of the electrocoagulation for area analysis with the reduction of noise in the sample.

FIGS. 6A-6E show tissue reactions after 1 MHz invasive bipolar radiofrequency (RF) treatment using insulated microneedle electrodes on in vivo porcine skin. In vivo porcine skin shows thermal injury in the dermis induced by radiofrequency with set parameters—1-MHz invasive bipolar RF, power level 4 (FIG. 6A), power level 5 (FIG. 6B), power level 6 (FIG. 6C), power level 7 (FIG. 6D), power level 8 (FIG. 6E), conduction time of 600-ms, penetration depth of 2-mm using a electrosurgical microneedling unit.

FIGS. 7A-J show tissue reactions after 2 MHz invasive bipolar RF treatment using insulated microneedle electrodes (FIGS. 7A-7E) and 1 MHz invasive bipolar RF using non-insulated microneedle electrodes (FIGS. 7F-7J) on in vivo porcine skin. In vivo porcine skin shows thermal injury similarities when using 2 MHz insulated microneedle electrode RF compared to 1 MHz non-insulated microneedle electrode RF in the dermis with set parameters—2-MHz (FIGS. 7A-7E) and 1-MHz (FIGS. 7F-7J) invasive bipolar RF, power level 4 (FIG. 7A, FIG. 7F), power level 5 (FIG. 7B, FIG. 7G), power level 6 (FIG. 7C, FIG. 7H), power level 7 (FIG. 7D, FIG. 7I), power level 8 (FIG. 7E, FIG. 7J), conduction time of 600-ms, penetration depth of 2-mm using a electrosurgical microneedling unit.

FIGS. 8A-8C show expected thermal temperature images under IR camera based off of electrode placement of MNRF system. FIG. 8A—Expected after 800 ms RF conduction time; FIG. 8B—Expected after a few seconds of thermal dispersion; FIG. 8—Expected thermal zones for MNRF system.

FIGS. 9A-9D show thermal temperature images of application area using insulated microneedle electrodes at RF conduction time of 800 ms and at Power Level 10. FIG. 9A—1 s; FIG. 10B—2 s; FIG. 10C—8 s; FIG. 10D—10 s

FIGS. 10A-10D show sinusoidal wave fit to temperature profile of radiofrequency microneedle. FIG. 10A—1 s; FIG. 10B—2 s; FIG. 10C—8 s; FIG. 10D—10 s.

### DETAILED DESCRIPTION

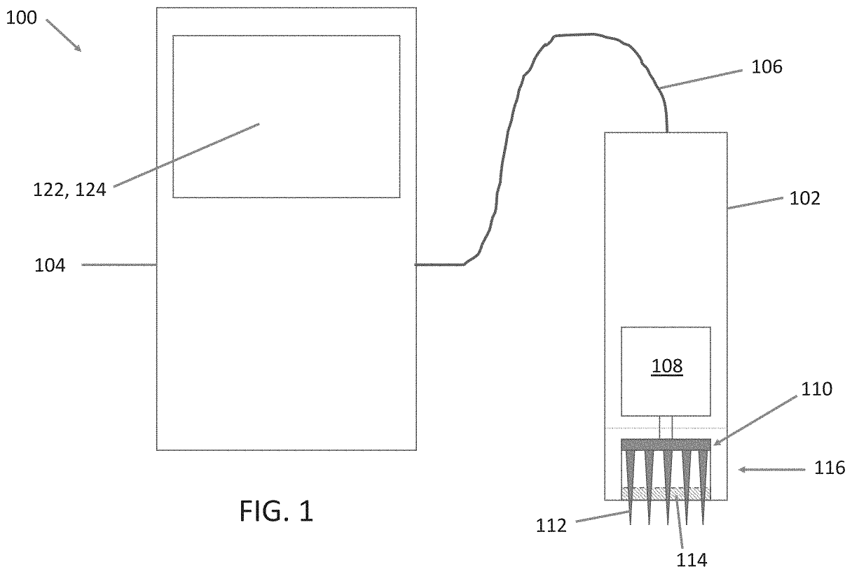

Disclosed herein is a microneedling system configured for providing a therapeutic treatment to a patient (e.g., a human patient). The microneedling system may be used to provide a cosmetic treatment, such as collagen induction therapy. FIG. 1 schematically depicts an example of a microneedling system 100. The microneedling system 100 may comprise a handpiece 102 configured to be held by the operator for applying the treatment to the patient's skin. In some embodiments, the handpiece 102 can be connected to a housing unit 104, which may be configured to control at least some operations of the handpiece 102 by a transmission line 106. The transmission line 106 may transfer power from the housing unit 104 to the handpiece 102. The transmission line 106 may communicate data or electrical signals between the housing unit 104 and the handpiece 102 (e.g., from the housing unit 104 to the handpiece 102 and/or from the handpiece 102 to the housing unit 104). In some embodiments, there may be no transmission line 106. The handpiece 102 may be battery-powered by a battery contained within the handpiece 102. In some embodiments, data and signals may be wirelessly communicated between the handpiece 102 and a housing unit 104 or a remote processor by any means known in the art.

The handpiece 102 may comprise a proximal end and a distal end. The handpiece 102 may comprise a generally elongate body extending between the proximal end and the distal end. The elongate body may be configured for grasping and handling by an operator. The transmission line 106 may extend from a proximal end of the handpiece 102. The handpiece 102 may comprise a microneedle array 110 for applying the microneedling treatment to the patient. The microneedle array 110 may comprise one or more microneedles 112 (e.g., 1, 2, 3, 4, 5, 6, 6, 8, 9, 10, 20, 30, 50, 60, 70, 80, 90, 100, or more than 100) extending from or configured to be extendable from the distal end of the handpiece 102. The microneedles 112 may be configured to penetrate the skin of the patient. The microneedles 112 may be used to induce collagen generation in the treated skin of the patient. The microneedles 112 may be arranged in a generally parallel fashion to each other such that the microneedles 112 are configured to be applied to penetrate the skin in a direction normal to the surface of the skin (e.g., when the elongate body of the handpiece 102 is held normal to the surface of the skin). The microneedles 112 may be arranged across a two-dimensional area of the microneedle array 110. In some embodiments, the microneedles 112 be arranged in rows and/or columns. The microneedles 112 may be uniformly spaced from each other in a regular pattern. The microneedles 112 may be configured to extend a uniform distance from the distal end of the elongate body such that the microneedles 112 may extend a uniform penetration depth into the skin of the patient. In some embodiments, one or more of the microneedles 112 may extend further (e.g., deeper) than one or more of the other microneedles 112. In some embodiments, one or more of the microneedles 112 may be configured to extend an adjustable depth into the skin of the patient when the distal end of the elongate body is held near or against the surface of the skin. For instance, in some implementations, the depth may be set between approximately 0.5 mm and 6 mm or between 0.5 mm and 3.5 mm. In some embodiments, the depth may be adjustable by 0.1, 0.2, 0.3, 0.4, 0.5, or 1 mm increments. In some embodiments, one or more of the microneedles 112 may have a diameter of no more than about 0.05 mm, 0.1 mm, 0.2 mm, 0.3 mm, 0.4 mm, or 0.5 mm. In some embodiments, the microneedles 112 may have a generally uniform diameter (e.g., circular) over the majority of the length of the needle, ending distally in a pointed tip. In some embodiments, the diameter may gradually taper over a distal portion of the length of the needle. In some embodiments, one or more of the microneedles 112 may have a length of no more than about 1 mm, 2 mm, 3 mm, 4 mm, 5 mm, 6 mm, 7 mm, 8 mm, 9, mm 10 mm, 11 mm, 12 mm, or 15 mm. In some embodiments, one or more of the microneedles 112 may be gold-plated. In some embodiments, one or more of the microneedles 112 may be hollow or made of an electrically nonconductive material that has a conductive coating. In some embodiments, one or more of the microneedles 112 may comprise an electrically conductive shaft or coating that is coated on the surface with an electrically non-conductive material. For example, an electrically non-conductive coating material can be patterned in order to chancel the radiofrequency energy to a particular location where the electrically conductive shaft or coating contacts the skin through a gap in the patterned electrically non-conductive coating. In some embodiments, one or more of the microneedles 112 may be insulated with insulation material.

In some embodiments, the microneedles 112 may be arranged in any suitable configuration to facilitate treatment. In some embodiments, the microneedles 112 may be substantially in a circular configuration. In some embodiments, the microneedles 112 may be in a circular configuration. In some embodiments, the microneedles 112 may be substantially in a rectangular configuration. In some embodiments, the microneedles 112 may be in a rectangular configuration. Other configurations may also be used if desired depending on treatment or application.

The handpiece 102 may comprise a motor 108 (e.g., a step motor or torque motor) for driving the microneedle array 110 in an oscillatory motion configured to puncture the skin of the patient. The motor 108 may drive each of the microneedles 112 simultaneously in a linearly reciprocating motion. In some embodiments, the microneedles 112 may be driven in a reciprocating motion in a temporally staggered fashion, such as in a linear (e.g., left-to-right) wave motion. The distal end of the handpiece 102 may be configured to be held against the skin of the patient prior actuating the microneedle array 110 such that the microneedles 112 are inserted a known depth into the skin when the motor 108 reciprocates the microneedle array 110. In some embodiments, the handpiece 102 may comprise a needle plate 114 through which the one or more microneedles 112 of the microneedle array 110 may extend during the skin puncturing motion. The needle plate 114 may comprise an aperture for each microneedle 112 through which the microneedle 112 may pass. The tip of the microneedle 112 may be positioned proximally behind or within the needle plate 114 such that the tips of the microneedles 112 are configured not to contact the skin in a resting state (e.g., prior to and/or after the microneedles 112 are reciprocated into the skin of the patient). The needle plate 114 may form a distal surface of the handpiece 102 such that the needle plate 114 may be placed into substantial contact with the skin during the treatment and the microneedles 112 may puncture and pass through the skin as they are extended distally beyond the needle plate 114 by the motor 108. In some embodiments, there may be no needle plate 114. In some embodiments, the handpiece 102 may comprise an opening at its distal end through which the tips of all the microneedles 112 may extend. The perimeter or rim of the opening may act as a contact surface which contacts the skin during treatment and positions the microneedles 112 a known distance from the skin prior to reciprocation. In some embodiments, the handpiece 102 may comprise an opening and a needle plate 114. The needle plate 114 may be positioned within the opening. The needle plate 114 may be positioned substantially flush with the edge of the opening or proximally positioned behind the opening. In some embodiments, the transmission line 106 may provide power to the motor 108 for driving the reciprocating motion. In some embodiments, the distal end of the handpiece 102 may comprise a detachable tip 116. The detachable tip 116 may enclose the microneedle array 110. The detachable tip 116 may be configured to detachably engage one or more pistons or other mechanical linkages extending from the motor 108. The detachable tip 116 may be configured for single-patient use (e.g., disposable) such that the detachable tip 116 can be replaced for different patients. The detachable tip 116 may be provided pre-sterilized in sealed packaging.

The microneedle array 110 may be configured to apply radiofrequency (RF) energy to the treated skin of the patient via an electric field. The term “RF energy” may be used to apply to energy delivered to the skin by electrodes of the system 100. In some implementations, other frequencies outside the radiofrequency spectrum may be suitable, in which case the term RF energy would be understood to nonetheless apply to energy delivered at those frequencies. The RF energy may be configured to heat confined volumes of the patient's skin (e.g., the dermis) to denature the proteins (e.g., the collagen) in the skin and induce a wound healing response that involves new collagen formation. Without being limited by theory, the RF energy may heat the tissue through the Joule effect, and the temperature achieved may depend in part on the resistivity of the heated tissue. The temperature reached by the application of RF energy may be configured to induce irreversible collagen coagulation, which may be ideal for promoting collagen regeneration. One or more of the microneedles 112 may be configured to serve as electrodes for delivering the RF energy to the skin. In some embodiments, all of the microneedles 112 may serve as electrodes. The puncture wound created by the microneedles 112 may induce wound healing and/or collagen regeneration. In some implementations, the effect between the puncturing and the thermal damage may be synergistic. In some embodiments, the microneedles 112 may be configured as monopolar electrodes in which all of the microneedle electrodes are configured as the same polarity (e.g., positive or negative) and the electrical charge delivered by the handpiece 102 is dissipated into the skin. The current may travel to a remote ground electrode not part of the handpiece 102. In some embodiments, the microneedles 112 may be configured as bipolar electrodes in which one or more of the microneedles 112 are configured as electrodes of a first polarity and one or more of the microneedles 112 are configured as electrodes of a second polarity, opposite the first polarity. Current supplied by the handpiece 102 may travel from the first polarity electrodes to the second polarity electrodes through the skin. The current may be an alternating current alternating at a frequency within the radiofrequency range such that the electrodes alternate between functioning as anodes and cathodes. Any other suitable frequency may also be employed. Bipolar stimulation of the skin may provide more confined and predictable volumes of treatment than monopolar stimulation. The electrode microneedles 112 may be either insulated or non-insulated. Non-insulated microneedles 112 may deliver the electrical current over the entire length of the microneedle 112. Insulated microneedles 112, may comprise a non-conductive covering over a proximal portion of the microneedle 112 which confines the delivery of the electrical current to the tip of the microneedle 112 (e.g., the distal most 0.1, 0.2, 0.3, 0.4, 0.5, 0.6, 0.7, 0.8, 0.8, or 1.0 mm). The collagen inductive effects may be greatest at the tip of the microneedles 112.

The microneedling system 100 may be configured to automatically deliver the RF energy during discrete time intervals. The time intervals may correspond to a period coinciding with the full insertion of each individual microneedle 112 through which the energy is delivered. In embodiments, in which the microneedles 112 are not all inserted simultaneously, corresponding electrodes (e.g., paired electrodes) through which an electric field is established may be configured to be inserted simultaneously or to at least be fully inserted during the duration of the RF energy pulse. In some embodiments, the electric current may be applied over one or more pulses of RF energy (e.g., 1, 2, 3, 4, 5, or more than 5 pulses) during each cycle of microneedle 112 insertion. In some embodiments, the pulse may be at least about 10, 50, 100, 200, 300, 400, 500, 600, 800, 900, or 1000 milliseconds in duration. The motor 108 may be configured to pause the reciprocating movement for a period of time equal to or greater than the duration of the one or more pulses, such that the microneedles 112 are not moving while they act as electrodes to deliver RF energy. In some embodiments, the total power delivered to the microneedle array 110 may be at least about 5, 10, 20, 30, 40, 50, 60, 70, 80, 90, or 100 W.

In some embodiments, the microneedling system 100 applies different RF waveforms. In some embodiments, the RF waveforms are modulated by pulsed, complex, or simple waveforms.

In some embodiments, one or more or all of the microneedles 112 may be substituted with surface electrode 113. The surface electrodes 113 may be in the form of probe electrodes (e.g., with flat or rounded distal tips) or conductive plate electrodes that are not configured to penetrate the skin. The surface electrodes 113 may be placed into contact with the surface of the skin and deliver RF energy from the surface of the skin (e.g., through the epidermal layer). For instance, in some embodiments, the microneedle array 110 may comprise a single electrode plate 113 for monopolar delivery of RF energy to the patient's skin. The system 100 may operate substantially the same as described elsewhere herein, excluding the modulation of penetration depth of the microneedle 112 electrodes. The RF energy will necessarily travel through superficial layers of skin (e.g., the epidermis) in passing through a surface electrode 113. Cooling treatments may be applied to the surface of the skin, as is known in the art, to avoid or mitigate damage to the superficial layers of the skin. In some embodiments, the surface electrodes 113 may be cooled to absorb heat from the surface of the patient's skin. The term “microneedle array” may also be used herein to refer to an electrode array comprising only microneedles 112, a combination of microneedles 112 and surface electrodes 113, or only surface electrodes 113.

The microneedles and/or the surface electrode(s) may be fabricated such that a material encloses one or more substrates that with adjusted frequency, current, voltage, material thickness, and/or conduction time can exhibit either non-insulated (conducting) properties or insulating properties for a specific or preselected distribution of energy. The energy can be on the electromagnetic spectrum, for example, radio frequency (20 kHz to 300 GHz, or roughly between the upper limit of audio frequencies and the lower limit of infrared frequencies), such as microwave (300 MHz to 300 GHz). The energy is dispersed to damage tissue, e.g., by coagulation. The material(s) that enclose the substrate(s) can be an amorphous material. The material that encloses the substrate can be a dielectric thin film. The material that encloses the substrate can be an oxide, e.g., one or more of a silicon oxide, a zirconium oxide, a lanthanum oxide, a lanthanum zirconium oxide or a cerium oxide. The material that encloses the substrate can be a polymeric material, e.g., a (substituted) paracyclophane, dichloro-di-p-xylylene, dichloro-paracyclophane, or trichloro-paracyclophane. The material that encloses the substrate can have a high thermal conductivity. The material that encloses the substrate can be selected based off of dielectric relaxation value corresponding to outputted frequency, current, voltage, material thickness, conduction time, or a combination thereof. The material that encloses the substrate can be a metal oxide semiconductor. The substrate can be a metal, e.g., iron, titanium, nickel, aluminum, or chromium, or a metal alloy, e.g., steel (e.g., stainless steel). The substrate can be in a form of a needle or in a form of a contact plate. An example of a suitable contact plate is one that can transition laser energy from fractional to nonfractional and vice versa. In one embodiment, a fractional pattern is provided by an array of apertures, e.g., holes or slots, or by a patterned array of emitters of laser energy. The pattern can be regular or irregular (random). Patterns and shapes as described elsewhere herein, e.g., in the context of arrays, can be adapted in regard to the fractional pattern.

The microneedling system 100 may comprise an interface 122 configured for interaction with an operator to operate the handpiece 102. The interface 122 may comprise one or more user inputs for incrementing, decrementing, or otherwise altering one or more operating variables of the microneedling system 100. The interface 122 may comprise one or more power and/or actuation switches for turning power to the system 100 and/or the handpiece 102 off/on and/or for initiating, pausing, stopping, restarting a function, such as motor oscillation, application of RF energy, and/or laser treatment. In some embodiments, in addition to or alternative to the control via the interface 122, one or more of these functions may be controlled by interface mechanism disposed on the handpiece 102 and/or on an implement at least somewhat remote from the housing unit 104, such as a foot switch operably coupled to the housing unit 104 or a keyboard or mouse operably coupled to a processor in communication with the housing unit 104 and/or the handpiece 102. The interface 122 may comprise buttons, switches, knobs, a keyboard, a mouse, and/or a touchscreen interface. The touchscreen interface may comprise widgets for receiving input from an operator. The microneedling system 100 may comprise a display 124 (e.g., a monitor screen) for displaying information to an operator. In some embodiments, the display 124 may function as a touchscreen interface forming at least part of the interface 122, as depicted in FIG. 1. The display 124 may be configured to display a graphical user interface (GUI) through which an operator can control the functioning of the microneedling system 100.

The microneedling system 100 may comprise one or more processors and/or memory. The processors and/or memory may be distributed between the housing unit 104, the handpiece 102, and one or more computing systems operably coupled to the housing unit 104 in any suitable combination or arrangement. The microneedling system 100 may comprise software for operating the system, including but not limited to controlling the motor 108 and/or controlling the power supplied to the RF electrodes. The software may be stored on memory within any one or more of the components of the system and/or stored on a remote server. The one or more processors may be in communication (e.g., wireless communication) with the servers for accessing the software. The memory may store one or more programs which may comprise preselected operating parameters and/or treatment protocols. In some embodiments, the programs may be stored on remote servers. The housing unit 104 may comprise a power source (e.g., a battery) and/or may be configured to couple to an external power source (e.g., a standard wall AC outlet).

One or more operating parameters of the microneedling system 100 may be adjustable. The adjustable operating parameters may be adjusted manually by the operator and/or automatically according to one or more algorithms, which may or may not depend on the selection of a stored treatment program. In some embodiments, one or more of the operating parameters may be set automatically unless manually overridden by the operator. For instance, the penetration depth of the microneedles 112, the frequency of the RF energy, the duration of the RF pulse, and/or the power of the RF energy may be modulated. In some implementations, the frequency, duration, and/or the power of the RF energy may be modulated to control the temperature and/or the size (e.g., volume) of the one or more coagulation volumes (via the electric fields) produced by the microneedles 112. Depending on the application, the targeted skin layer, the thickness of the targeted skin layer, and/or the proximity of adjacent skin layers, larger or smaller volumes of coagulation may be desired. The optimal temperatures achieved within the tissue resulting from the electric field applied may also depend on the application and the tissue being treated.

In some embodiments, the power level may be selectable from an arbitrary incremented scale (e.g., 1 to 10, 1 to 5, etc.). The voltage/current and frequency of the signal may affect the amount of energy delivered to the tissue. For instance, higher frequencies of alternating current (having shorter wavelengths) may deliver higher amounts of energy. Higher voltages and/or higher currents (the prospective amplitudes of an alternating current waveform) may deliver higher amounts of energy. The voltage and current may be related to each other according to Ohm's law (V=IR) depending on the resistivity of the treated tissue. In various implementations, the frequency and/or the voltage/current of the RF energy may be modulated to adjust the amount of power or energy delivered to the tissue. In some embodiments, the frequency may be adjustable between approximately 1 kHz and 100 MHz, between 100 kHz and 50 MHz, and/or between 0.5 MHz and 10 MHz. For example, a higher power level (e.g., level 10) may utilize a frequency of about 2 MHz and a lower power level (e.g., level 5) may use a frequency of about 1 MHz. Some levels may employ the same frequency but have varying amplitudes of voltage/current. In some embodiments, the processor may employ one or more algorithms for determining the appropriate frequency and voltage/current combination to deliver the desired amount of energy. For instance, in some implementations, increasing the energy through higher frequency or higher current/voltage may differentially affect the amount of pain perceived by the patient.

In some embodiments, the microneedling system 100 may include a temperature sensor (not shown). The temperature sensor may be added to ensure the microneedles 112 are not overheating the treated skin layer above the desired therapeutic temperature. Some embodiments may further include a cutoff feature that prevents the microneedling system 100 from heating above a certain temperature. In some embodiments, the temperature is set by a clinician. In some embodiments, the temperature is preset by the manufacturer.

In some embodiments, the microneedling system 100 may include a cooling apparatus (not shown). In some embodiments, the cooling apparatus cools a desired area prior to treatment. In some embodiments, the cooling apparatus may be applied to the treated area sustainably concurrently with treatment. In some embodiments, the cooling apparatus may be applied after treatment to an area of skin. In some embodiments, the cooling apparatus is a thermoelectric cooling plate. Such embodiments provide maximum comfort to the patient as well as providing practitioners with way to provide an optimal or high-powered treatment to the patient without damaging skin or causing discomfort, thereby improving the likelihood of a desirable outcome.

In some embodiments, the microneedling system 100 may further comprise a camera or visual observation device (not shown). In some embodiments, the microneedle device may include an IR camera. In some embodiments, the IR camera can be used to identify or visualize the area to be treated within the skin. In some embodiments, the IR camera may be used for temperature feedback. For example, the temperature may be taken during application of the RF energy from inside the skin layer or outside the skin layer. In another example, the top of the skin may be used for a temperature gradient or thermal profile that accounts for on or off times. In some embodiments, a thermocouple may be used within one or more microneedle 112.

In some embodiments, the microneedling system 100 may provide a feedback signal related to an electrode impedance, a bioimpedance, a local electrical field, and/or an electrophysiological response to the radiofrequency signal. In some embodiments, the bioimpedance may be related to temperature rising in the layer of skin. In some embodiments, the bioimpedance may be related to the water loss caused by the rising temperature. In some embodiments, the bioimpedance may be thermal stress. In some embodiments, the bioimpedance may be thermal damage. In some embodiments, the bioimpedance may be cell damage. In some embodiments, the bioimpedance may be protein damage. In some embodiments, the bioimpedance may be heat shock. In some embodiments, the bioimpedance may be a perturbation of the physiological characteristics of the target skin layer.

In some embodiments, the microneedling system 100 may further comprise an ultrasonic element (not shown). In some embodiments, the microneedling system 100 may comprise two or more ultrasonic elements. In some embodiments, the ultrasonic element may be an ultrasonic generator. In some embodiments, the ultrasonic element has an ultrasound frequency between about 20 MHz to about 200 MHz. In some embodiments, the ultrasonic element has an ultrasound frequency between about 50 MHz to about 200 MHz. In some embodiments, the ultrasonic element has an ultrasound frequency between about 50 MHz to about 100 MHz. In some embodiments, the ultrasonic element has an ultrasound frequency between about 100 MHz to about 200 MHz. In some embodiments, the ultrasonic element produces high intensity focused ultrasound. In some embodiments, the ultrasonic element is an ultrasonic transducer. In some embodiments, the ultrasonic element is an acoustic transducer. In some embodiments, the ultrasonic element produce acoustic waves to treat the target skin layer with Acoustic Wave Therapy. In some embodiments, the Acoustic Wave Therapy and radiofrequency treats cellulite.

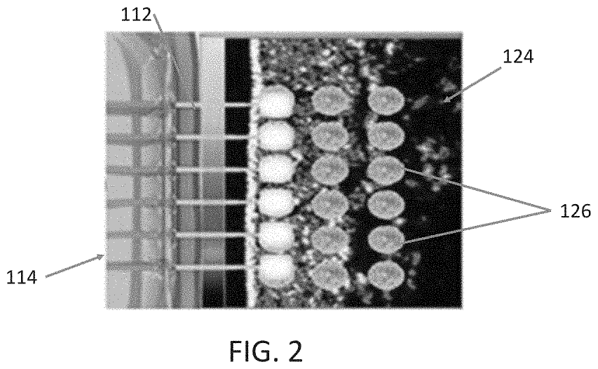

FIG. 2 schematically depicts an image of the display 124 in which a plurality of coagulation volumes 126 have been visually depicted on a representative image of the skin. FIG. 2 also shows an image or depiction of the distal end of the handpiece 102 overlaid on the representative image. In some embodiments, the display 124 may show or otherwise depict the relative positioning of the microneedle array 110 or at least one or more of the microneedles 112 on the image of the tissue. The microneedles 112 may be shown over the skin surface and/or penetrating the tissue at any possible penetration depth achievable by the microneedling system 100. The size and/or shape of the volumes of coagulation may be predicted based on algorithms which may account for the operating parameters and/or the thickness of the substituent layers of the skin. The display may depict expected widths, volumes, temperatures, and/or separation distances of the volumes of coagulation 126. An operator may make further adjustments based on the expected characteristics of the volumes of coagulation 126. In some embodiments a representative volume of coagulation (e.g., an average volume of coagulation) may be input by the operator and/or predetermined by a stored program. The needle penetration depth, the pulse duration, the frequency, and/or the power may be adjusted according to the representative volume of coagulation alone or in combination with other inputs. As shown in FIG. 2, the display 124 may depict the volumes of coagulation 126 at different depths according to the variable penetration depth of the microneedles 112. In some embodiments, the microneedling system 100 may penetrate the same area of interest repeatedly with microneedles 112 using different penetration depths to result in a three-dimensional array of volumes of coagulation 126 as shown in FIG. 2. The remaining operating parameters may be the same or different for each penetration depth. In some embodiments, the microneedling system 100 may be configured to rapidly perform multiple penetrations at different depths before the operator substantially moves the handpiece 102 to a different area of skin. For instance, the motor 108 may cycle between two or more penetration depths in a regular pattern. In some embodiments, multiple passes may be made over the same treatment area with the penetration depth being different for each pass. The microneedling system 100 may modulate the penetration depth according to any of these protocols based on a preselected algorithm.

The microneedle array 110 may comprise any suitable arrangement of electrodes 115. In non-monopolar embodiments, the array 110 may comprise electrodes 115 of opposite polarity such as positive electrodes 115a and negative electrodes 115b. Electrodes 115 may comprise any combination of microneedles 112 and/or surface electrodes 113 unless otherwise specified. The array 110 may comprise a microneedles of a positive polarity 112a and/or microneedles of a negative polarity 112b. The array 110 may comprise surface electrodes 113 of a positive polarity 113a and/or surface electrodes of a negative polarity 113b. In some embodiments, one of the polarities (e.g., the negative polarity electrodes 115b) may be at ground reference voltage. It is to be understood that in the various embodiments disclosed herein the polarities of the electrodes 115 may be switched and the system 100 may operate in substantially the same fashion unless otherwise specified. In some embodiments, the array 110 may have a round (e.g., circular) or a polygonal area (e.g., triangular, square, pentagonal, hexagonal, octagonal, etc.) across which the electrodes 115 are arranged. The electrodes 115 may be arranged substantially uniformly across the area of the array 110. In some embodiments, the array 110 may comprise a surface area of at least about 0.1, 0.2, 0.3, 0.4, 0.5, 0.6, 0.7, 0.8, 0.9, 1.0, 1.25, 1.5, 1.75, 2.0, 2.5, 3, 4, 5, or more than 5 cm².

In embodiments comprising bipolar electrodes, the RF energy may be substantially confined to discrete fields between one or more electrodes of a first polarity and one or more electrodes of a second polarity, opposite the first polarity. The confined fields may form volumes of coagulation in which the tissue temperature within the volume is sufficiently raised to a degree sufficient to induce collagen coagulation, as described elsewhere herein. In some implementations, collagen may begin to denature at about 40-48 degrees Celsius. Collagen may coagulate at about 55-70 degrees Celsius. Higher temperatures may require shorter durations of heating to induce coagulation or permanent denaturation. The area or volume of raised temperature (e.g., coagulation volume) may expand over the duration of heating. The area or volume of raised temperature may expand more rapidly at higher temperatures. The microneedle array 110 may produce one or more volumes of coagulation. Each volume may be shaped by the electric field between proximate microneedles 112 of opposite polarity. In some embodiments, each volume may be generally spherical or ellipsoid. In some implementations, one or more of the volumes of coagulation may merge together forming a combined volume of coagulation in which the constituent volumes are indistinguishable. In some implementations, one or more of the volumes of coagulation may touch or overlap, such that each of the constituent volumes remains distinguishable from the others. In some implementations, one or more of the volumes of coagulation may remain sufficiently isolated such that there is no overlap or contact between the volumes. For a given arrangement of microneedles 112 of a microneedle assay 110, maintaining the volume of one or more volumes of coagulation below threshold levels may keep the volumes separate and distinct such that they are separated by regions of tissue in which collagen is not coagulated and the tissue remains relatively unwounded. In some implementations, this allows delivering the RF energy in a fractionated manner, in which wounded volumes of tissue are surrounded by healthy tissue. Fractionating the RF energy may advantageously promote the wound-healing response, reduce patient pain, shorten the recovery time, and/or reduce the likelihood of infection. Without being limited by theory, more uniform distribution of untreated areas within treated regions of skin may more advantageously promote the healing response and/or improved collagen induction in the treated areas. The untreated skin may promote more rapid wound healing of adjacent coagulation volumes. The minimization in the total coagulation volume may result in less pain to the patient and/or a more rapid recovery time.

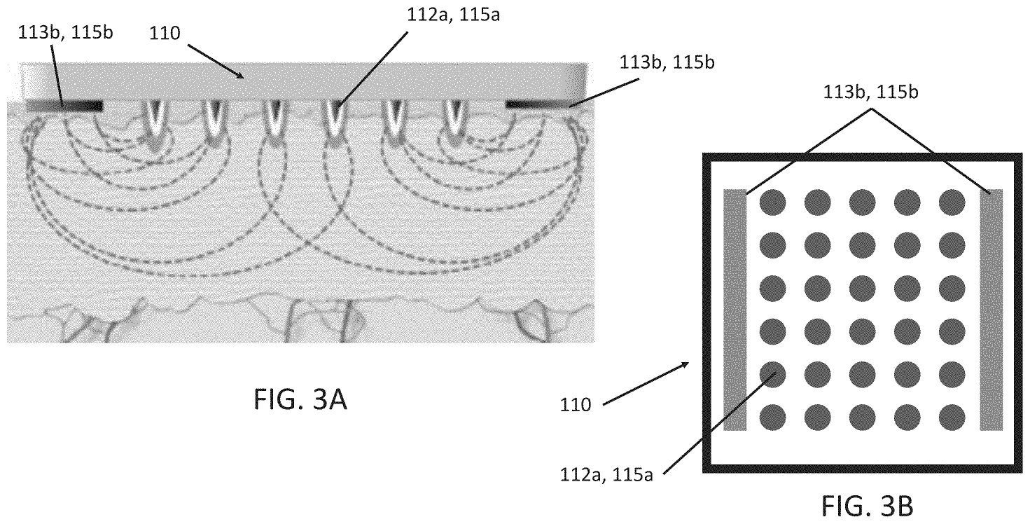

FIGS. 3A and 3B schematically illustrate an example of an arrangement in which a plurality of positive microneedles 112a are disposed within a central region of the array 110 and two negative (ground) electrode plates 113b are disposed on opposite lateral edges (e.g., right and left edges) of the array 110. FIG. 3A depicts a profile view of the array 110 in contact with the skin of a patient including positive microneedles 112a extending into the skin (just beyond the epidermis into the dermis) and negative surface electrode plates 113b pressed into contact with the surface of the skin. FIG. 3B depicts a surface view of the microneedle array showing the 2-dimensional arrangement of positive microneedles 112a and negative surface electrode plates 113b. As show by the dashed lines in FIG. 3A representing electric field lines between positive microneedles 112a and negative surface electrodes 113b, the energy delivered through the positive microneedles 112a will exclusively or primarily travel to the closest negative electrode 113b assuming substantially equal resistivity of the tissue below the array 110. The energy delivered through the positive microneedles 112a disposed on the left portion of the microneedle array 110 will tend to travel through the tissue to the left negative electrode 113b, while the energy delivered through the positive microneedles 112a disposed on the right portion of the array 110 will tend to travel through the tissue to the right negative electrode 113b. The tissue may experience a cumulative effect of the overlapping electric fields. An electric flux and/or temperature gradient may be formed extending from a central midline of the microneedle array 110 where the temperature and/or electric flux is the lowest to the right and/or left sides of the array 110 where the temperature and/or electric flux is the highest. Bulk heating may be experienced towards one or both of the left and right sides of the array 110. The left and/or right sides may be heated to above an ideal temperature and/or a central region may be heated to below an ideal temperature. The gradients may be symmetric. If the tissue beneath the array 110 is not homogenous, the gradients may be asymmetric. For example, if the tissue below either the left or right portion of the microneedle array 110 has a larger resistivity than the opposite side, then less or no current may travel through the more resistive portion. Energy from positive microneedles 112a on the left portion of the array 110 may travel to the right negative surface electrode 113b or vice-versa, resulting in asymmetric heating and likely achieving higher than ideal temperatures on the less resistive side and/or lower than ideal temperatures on the more resistive side. In some instances, differences in tissue resistivity may be caused by anatomical features such as glands, pores, etc. Because the electric fields emanating from the positive microneedles 112a overlap to reach the negative surface electrodes 113b, fractionating of the RF energy may be impossible or diminished. The effects of the arrangement shown in FIGS. 3A-3B may be the same if any or all of the positive microneedles 112a are substituted with positive surface electrodes 112b and/or if either of the negative surface electrodes 113b are substituted with a plurality of negative microneedles 112b. Similar effects may be observed to varying degrees for any arrangement in which the path between a positive electrode 115a and the closest negative electrode 115b extends across another positive electrode 115a.

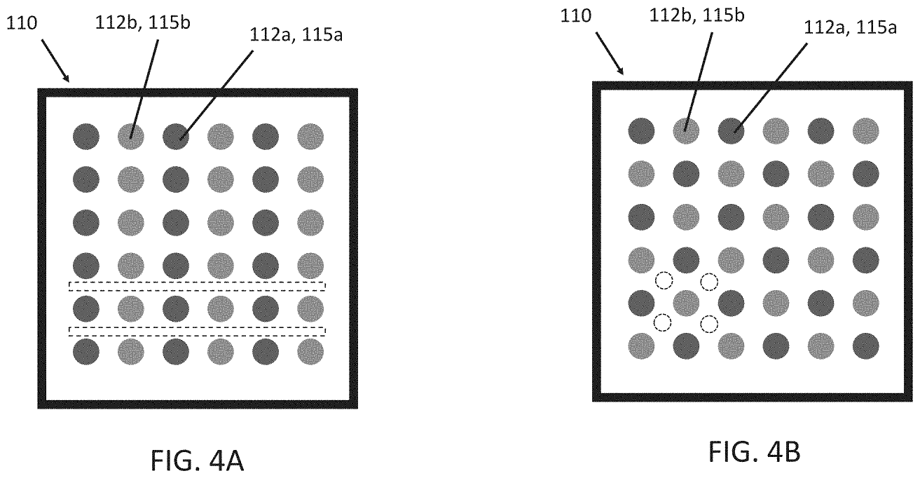

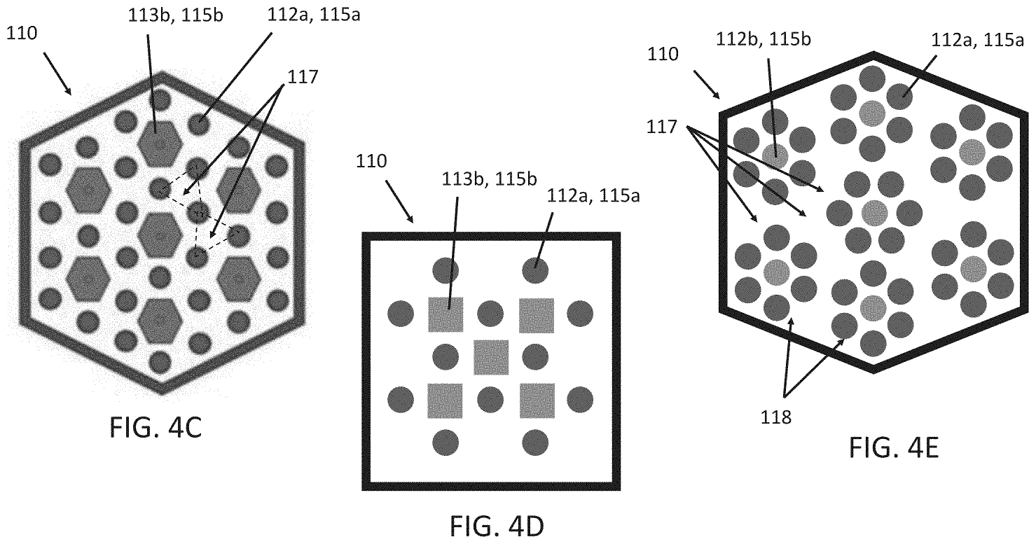

FIGS. 4A-4E depict alternative arrangements of electrodes 115 on an array 110. In some embodiments, the array 110 may comprise an arrangement of positive electrodes 115a and negative electrodes 115b in which the negative electrodes 115b are substantially uniformly dispersed amongst the array of positive electrodes 115a. The microneedle array 110 may be arranged such that each positive electrode 115a is positioned adjacent a negative electrode 115b. In some implementations, a positive electrode 115a may be considered adjacent a negative electrode 115b if no other electrodes 115 are disposed between the positive electrode 115a and negative electrode 115b. In some embodiments, all positive electrodes 115a may be spaced a substantially equal distance from the closest negative electrode 115b. In some embodiments, the electrodes 115a may be arranged in a uniform pattern. For example, the electrodes 115 may be arranged along evenly spaced rows and/or evenly spaced columns. The electrodes 115 may be arranged on a square grid or other regular latticed coordinate system. By arranging the array 110 in a manner that maximizes the number of electrodes 115 which are adjacent an electrode 115 of opposite polarity the travel path of energy between electrodes 115 may be effectively reduced, minimized, or otherwise optimized. Reducing the travel path of the RF energy to the closest electrode 115 of opposite polarity may provide more control over delivery of the energy and/or may reduce the amount of pain or discomfort experienced by the patient. In some implementations, RF energy may travel only or primarily between an electrode 115 (e.g., a positive electrode 115a) and the closest electrode 115 of opposite polarity (e.g., a negative electrode 115b). The energy may flow more predictably over shorter distances. Heterogeneities in the tissue may be less likely to intervene these pathways and/or the effects may be mitigated over shorter distances, particularly where the next closest electrode 115 of the opposite polarity is a significantly further distance away. In some embodiments, the electrodes 115 may be positioned no further than 0.1, 0.2, 0.3, 0.4, 0.5, 0.6, 0.7, 0.8, 0.9, 1.0, 1.5, 2.0, 2.5, 3.0, 3.5, 4.0, or 5.0 mm from the closest adjacent electrode 115 of opposite polarity.

FIGS. 4A and 4B depict examples of arrays 110 in which electrodes 115 are arranged on a square grid. The electrodes 115 may be arranged in a manner such that each positive electrode 115a is adjacent a negative electrode 115b, as shown in FIGS. 4A and 4B. The electrodes 115 may be arranged in a manner such that all the positive electrodes 115a are spaced a substantially equal distance from the closest negative electrode 115b, as shown in FIGS. 4A and 4B. The arrays 110 in both FIGS. 4A and 4B comprise a 1:1 ratio of positive electrodes 115a to negative electrodes 115b. The electrodes 115 depicted in FIG. 4A are arranged in alternating columns of positive electrodes 115a and negative electrodes 115b. Except for electrodes 115 on the right and left edges, which are adjacent exactly one closest electrode 115 of the opposite polarity, each electrode 115 is adjacent two closest electrodes 115 of the opposite polarity. The electrodes 115 depicted in FIG. 4B are arranged in a checkerboard fashion. Corner electrodes 115 are adjacent exactly two closest electrodes of the opposite polarity. Other electrodes 115 on the edge of the array 110 are adjacent exactly three closest electrodes 115 of the opposite polarity. All other electrodes 115 are adjacent exactly four closest electrodes 115 of the opposite polarity. The RF energy delivered from the positive electrodes 115a may fractionate to the closest negative electrode 115b. Where a positive electrode 115a is positioned a substantially equal distance from multiple negative electrodes 115b, the energy may fractionate between the positive electrode 115a and the plurality of negative electrodes 115b in a substantially equal fashion. Maximizing the number of positive electrodes 115a, which are adjacent a closest negative electrode 115b may generally achieve more uniform heating across the entire area of the array 110. In some implementations, increasing the total number of closest negative electrodes 115b adjacent a single positive electrode 115a and/or increasing the total number of closest positive electrodes 115a adjacent a single negative electrode 115b may more uniformly distribute the RF energy across a localized area of tissue around the single electrode 115. For example, the RF energy may be more uniformly distributed across the array 110 in FIG. 4B than in FIG. 4A because the electrodes 115 tend to be adjacent to a larger number of closest opposite polarity electrodes 115. In some embodiments, adjacent to can mean that two structures are next to each other without any structure in-between the two structures.

In some arrangements, particularly those comprising a 1:1 ratio of positive electrodes 115a to negative electrodes 115b, there may be a tradeoff between uniform distribution of energy across a localized area and fractionating the energy delivery over the total area of the array 110 such that some areas or zones are left effectively untreated, which may enhance the wound healing response as described elsewhere herein. The arrangement of electrodes 115 may be used to modulate the total area (e.g., percentage of area) of treated skin that is effectively left untreated. There may be an optimal ratio of treated to untreated areas which optimizes the healing response (e.g., reduces healing time) and/or reduces the chance of infection and/or minimizes patient pain. The substantially uniform distribution of untreated zones within treated tissue may improve the wound healing response. The arrangement of electrodes 115 may result in zones 117 across the array 110 in which energy is not effectively delivered or in which a relatively negligible amount of energy is delivered. Example of possible untreated zones 117 are depicted in FIGS. 4A and 4B. As shown in comparison of FIGS. 4A and 4B, the more uniform spatial distribution of the RF energy in FIG. 4B may effectively diminish the cumulative area of the zones 117. Although depicted in ordinary geometric shapes in FIGS. 4A-4E, the borders of the zones 117 may be more complex and/or less definitively defined. However, the size or existence of the zones 117 may depend on the span of the electric flux between electrodes 115 of opposite polarity. In some embodiments, RF energy that is delivered at higher power may have a greater expanse across the treated tissue diminishing or altogether eliminating one or more of the untreated zones 117.

In some embodiments, the array 110 may comprise an unequal number of positive electrodes 115a and negative electrodes 115b. For instance, the array 110 may comprise a 2:1, 3:1, 4:1, 5:1, 6:1, 7:1, 8:1, 9:1, 10:1 ratio, a ratio in a range defined there between, or a more than 10:1 ratio of positive electrodes 115a to negative electrodes 115b or vice-versa. The higher the ratio, the more majority-type electrodes 115 may be positioned adjacent to the closest minority-type electrodes 115. For instance, in an arrangement comprising 6:1 positive electrodes 115a to negative electrodes 115b, each negative electrode 115b may be arranged to be adjacent to six closest positive electrodes 115a. The uniform distribution of six positive electrodes 115b clustered around, grouped around, or adjacent to a single negative electrode 115b may establish a more uniform electric field centered around the negative electrode 115b than between a single positive electrode 115a and single negative electrode 115b. Higher ratios of positive to negative electrodes 115a:115b may allow arrangements that maximize the number of closest positive electrodes 115a adjacent to a negative electrode 115b and minimize the number of closest negative electrodes 115b adjacent to a positive electrode 115a. Higher ratios of negative to positive electrodes 115b:115a may allow arrangements that maximize the number of closest negative electrodes 115b adjacent to a positive electrode 115a and minimize the number of closest positive electrodes 115a adjacent to a negative electrode 115b. Arrangements with a high number of positive electrodes 115a adjacent to a closest negative electrode 115b may uniformly distribute electric fields around the negative electrodes 115b, while arrangements having a simultaneous low number of closest negative electrodes 115b adjacent to each positive electrode 115a may effectively isolate the electric field emanating from the positive electrodes to discrete areas around the negative electrodes 115b, which can improve fractionation of the RF energy over the array 110.

FIG. 4C depicts an example of an array 110 comprising a generally hexagonal arrangement of electrodes 115. The electrodes 115 shown in FIG. 4C comprise positive microneedles 112a and negative surface electrodes 113b. Each negative electrode 115b is uniformly surrounded by six adjacent closest positive electrodes 115a. The negative electrodes 115b may share closest adjacent positive electrodes 115a, as shown in FIG. 4C, such that some positive electrodes 115a are positioned adjacent two negative electrodes 115b. In other embodiments, some positive electrodes 115a may be shared such that they are adjacent more than two closest negative electrodes 115b (e.g., adjacent, 3, 4, 5, or more negative electrodes 115b). The remaining positive electrodes 115a are adjacent only one closest negative electrode 115b. In some embodiments, one or more of the electrodes 115, particularly the surface electrodes 113, may comprise a non-circular or non-round cross-section. In some embodiments, the electrode 115 may comprise a polygonal cross section (e.g., 3, 4, 5, 6, 7, 8, 9, or more sides). The polygon may be a regular polygon, being equiangular and equilateral. The number of sides of the polygon may correspond to the number of adjacent electrodes 115 of opposite polarity. For instance, as seen in FIG. 4C, the array 110 comprises hexagonal negative surface electrodes 113b, which are each centered within six adjacent positive microneedles 112a. The closest adjacent electrodes 115 may be positioned adjacent a flat edge of the polygon, as shown in FIG. 4C, or adjacent a corner of the polygon. As shown in FIG. 4C, the arrangement may result in untreated zones 117 that are not in the path between any pairs of closest positive electrodes 115a and negative electrodes 115b, such as the triangular regions positioned between three closely spaced positive electrodes 115a. These zones 117 may provide areas of untreated tissue that allow fractionation of the RF energy over the total area of the array 110. The size of the zones 117 may be modulated by the spacing of the electrodes 115 to increase or decrease the total area of untreated skin. The zones 117 depicted in FIG. 4C may be relatively stable compared to those depicted in FIGS. 4A and 4B because they are defined at the zone borders by electrodes 115 of a single polarity rather electrodes of opposite polarity. FIG. 4D depicts another example in which square negative surface electrodes 113b are each surrounded by four closest adjacent positive microneedles 112a.

In some embodiments, a central electrode 115 or electrode 115 which is surrounded by a plurality of closest adjacent electrodes 115 of opposite polarity, particularly if the plurality of opposite polarity electrodes 115 comprises a higher number of electrodes 115, may comprise a larger surface diameter and/or cross-sectional area. For instance, the cross-sectional area of each negative surface electrode 113b in FIG. 4C may be substantially larger than the cross-sectional area of each closest adjacent positive microneedle 112a. Larger cross-sectional areas may more uniformly distribute the electric field and/or heating across a localized area. In some embodiments, the cross-sectional area of an electrode 115 may be at least about 1.25, 1.5, 2, 3, 4, 5, 6, 7, 8, 9, 10, or more than 10× greater than that of a closest adjacent electrode 115 of opposite polarity. Surface electrodes 113 (e.g., plate electrodes) may be particularly suitable for increasing the surface area of the electrode 115. Increasing the area of the surface electrode 113 may decrease patient pain and/or damage to the superficial layers of skin (e.g., the epidermis). The use of surface electrodes 113 may decrease patient pain or discomfort by reducing the total number of microneedles 112 that need to be inserted into the patient skin while maintaining a particular number of electrodes 115. The combination of microneedles 112 and surface electrodes 113 may combine the advantages of each, such as large cross-sectional areas and/or no penetration of surface electrodes 113 and the targeted depth and/or stimulating effect of microneedles 112. The use of only or predominantly microneedles 112 may advantageously prevent or reduce epidermal heating and damage, particularly if insulated microneedles 112 are employed.

FIG. 4E depicts an example of an array 110 comprising a plurality of electrode islands 118. The islands 118 shown in FIG. 4E are composed of negative microneedles 113b surrounded by positive microneedles 112a. In some embodiments, the array 110 may comprise 2, 3, 4, 5, 6, 7, 8, 9, 10, or more than 10 islands 118 of electrodes 115. The islands 118 may comprise at least one positive electrode 115a and at least one negative electrode 115b. The electrodes 115 of an island 118 may be effectively electrically isolated from electrodes 115 outside the island 118 by arranging the electrodes 115 of the array 110 such that all of the electrodes 115 inside the island 118 are closer to an electrode 115 of opposite polarity inside the island 118 than an electrode 115 of opposite polarity outside the island 118 and such that all the electrodes 115 outside the island 118 are closer to an electrode 115 of opposite polarity outside the island 115 than an electrode 115 of opposite polarity inside the island 118. Assuming relatively homogenous tissue resistivity, electric current will tend not to travel between the island 118 and electrodes 115 outside the island 118. In some embodiments, all the electrodes 115 of an array 110 may be distributed into islands 118, as shown in FIG. 4E. The islands 118 may be arranged in a uniform pattern. The pattern may or may not resemble an arrangement of individual electrodes 115 within the islands 118. The islands 118 may comprise the same or different arrangements of electrodes 115. In some embodiments, the islands 118 may comprise identical orientations. In some embodiments, the islands may comprise different orientations as shown in FIG. 4E, where the central island 118 is oriented in a different direction than the surrounding islands 118. Islands 118 may promote fractionation of the RF energy by eliminating or minimizing the presence of electric fields between the individual islands 118. The tissue between the islands 118 may be effectively untreated, and thus, may improve the wound healing response as described elsewhere herein. Accordingly, all the area or substantially all the area between the individual islands may comprise an untreated zone 117. The tissue inside the island 118 (encompassed within an area defined by the electrodes 115 forming the edge or border of the island 118) and, in some implementations, near the islands 118 may be treated by the RF energy. The arrangement of unequal ratios of positive and negative electrodes 115a, 115b on an island 118 may promote uniform distribution of energy across the tissue inside the island 118 as described elsewhere herein. The spacing of electrodes 115 on the island 118 closer to each other and/or the spacing of electrodes 115 outside the island 118 further from the island 118 may make the electric fields within the island more predictable by mitigating the effects of tissue heterogeneity under the array 110. In some embodiments, an island 118 may be separated from outside electrodes 115 by a distance greater than the distance separating the electrodes 115 on the island 118. In some embodiments, the electrodes 115 of the island may be separated from electrodes outside the island by the same distance that separates electrodes 115 on the island 118, but the electrodes 115 outside the island 118 which are positioned closest to the electrodes 115 inside the island 118 may be the same polarity as those electrodes 115 forming a border of the island 118, or at least to the closest electrode 115 within the island 118, such that current does not tend to travel between these electrodes 115 inside of and outside of the island 118. In some embodiments, the islands 118 may be positioned at least 0.1, 0.2, 0.3, 0.4, 0.5, 0.6, 0.7, 0.8, 0.9, 1.0, 1.5, 2.0, 2.5, 3.0, 3.5, 4.0, or 5.0 mm from the closest adjacent island 188 (e.g., measured from border-to-border). The various features of the arrangements of the microarrays 110 disclosed herein may be combined in any suitable manner.

In some embodiments, the handpiece 102 may comprise one or more sources of laser light for treating the patient's skin. For instance, the handpiece 102 may comprise one or more LEDs (laser emitting diodes) configured to produce light of specific wavelengths for providing therapeutic treatment to the skin. For example, a blue LED light may be used to disrupt bacterial growth. A red LED light may be used to stimulate collagen production. The source of light may be positioned laterally to the microneedle array 110 and/or may be positioned proximally behind the microneedle array 110. In some embodiments, components of the handpiece 102 positioned distally to the light source, such as possibly the microneedle array 110 and/or the needle plate 114 may be translucent or at least partially translucent to the wavelengths of light transmitted by the light source such that light may pass distally through the distal end of the handpiece to the patient's skin. In some implementations, photonic stimulation may be provided through a separate instrument or handpiece to supplement the microneedling treatment.

In some implementations, the ideal penetration depth of the microneedles 112 may depend on the thickness of the skin, the desired effects of the treatment, and/or the particular area of the body where the skin is being treated. For instance, an ideal penetration depth for treatments designed to tighten the skin via collagen induction may be one which reaches the dermis layer of the skin. In some implementations, the lower (deeper) layers of the dermis may be the optimal target depth for the tips of the microneedles 112. An ideal penetration depth for treatments designed to treat superficial scarring of the skin (e.g., acne scarring) may be one which does not surpass the epidermal layers of the skin or which only reaches very superficial layers of the dermis. In some implementations, the subcutaneous layers may be avoided. Avoiding the subcutaneous layers may avoid excessive damage, bleeding, and/or pain to the patient. The thickness of the skin, particularly the thickness of the epidermis layers may vary from patient-to-patient and/or depending on the location of the skin on the patient (e.g., the forehead, cheeks, neck, stomach, etc.). For instance, the epidermis may be much thinner on the neck of a patient than on the cheeks of a patient. Also, the dermis may become thinner with age or exposure to other environmental factors, such as UV radiation. The amount of fat deposits between patients may vary and effect the thicknesses of one or more layers of skin. For instance, a patient's Body Mass Index (BMI) may be correlated to skin thickness. Accordingly, the optimal microneedle 112 penetration depth may depend on the thickness of the skin of a precise treatment area for a particular patient as well as on the specific results to be achieved.

In some embodiments, the microneedling system 100 creates a uniform area of heating. As used herein, “substantially uniform” or “substantially uniform area of heating” is defined as a heat transfer provided by a microneedling system 100 having a substantially uniform temperature profile. In some embodiments, the uniform area of heating is substantially uniform. In some embodiments, the uniform area of heating varies between 0.5 degrees Celsius to about 5 degrees Celsius at the site of application. In some embodiments, the uniform area of heating varies by 0.5, 1.0, 1.5, 2.0, 2.5, 3.0, 3.5, 4.0, 4.5, and 5.0 degrees Celsius, or any range or value inbetween. In some embodiments, the therapeutic temperature is between about 40 degrees Celsius to about 70 degrees Celsius. In some embodiments, the therapeutic temperature is between about 50 degrees Celsius to about 70 degrees Celsius. In some embodiments, the therapeutic temperature is between about 60 degrees Celsius to about 70 degrees Celsius.

In some embodiments, the electrodes may be configured to form a uniform RF treatment. In some embodiments, the electrodes drive the uniform RF treatment to form a uniform current distribution between the electrodes. In some embodiments, the skin treatment can be self limiting to create treatment zones of approximately uniform size across the uniform treatment pattern.

### Examples

Radiofrequency, originally used just for radio-wave communication, is a frequency range in the electromagnetic spectrum that is a high frequency alternating current that is now used in combination with microneedling. Radiofrequency devices are used for a variety of dermatologic conditions and are found in dermatology, plastic surgery, dental, obstetrics and gynecology, and typical medical spa offices. These devices have been used to treat several dermatological conditions such as wrinkles, acne scars, enlarged pores, and acne vulgaris. Radiofrequency is delivered in two modalities: mono-polar and bi-polar. Mono-polar radiofrequency involves two poles that are spaced apart, a considerable distance, where one of the poles is usually a negative ground plate and the other pole is usually the applicator plate that establishes the radiofrequency. Mono-polar radiofrequency has very little control of what skin layers the user is applying the radiofrequency and thus may cause thermal damage in undesired areas. Bi-polar radiofrequency involves two or more active electrodes where one electrode is positive and one is negative where the RF travels to one electrode to the other establishing a target area for the treatment. The main difference between mono-polar and bi-polar is that mono-polar is pole-pole and bi-polar is electrode-electrode. The main advantage of using bi-polar rather than mono-polar is that using electrode-electrode configurations the user has the opportunity to establish thermal injury in a specific skin-layer rather than the mono-polar modality where the user would usually have very little control over the injury area.

Microneedling radiofrequency (MNRF) uses a bi-polar configuration where the electrodes are configured as positive and negative array for optimal treatment area. MNRF was first established by David Utely in Systems and Methods for Shrinking Collagen in the Dermis (U.S. Pat. No. 6,277,116 B1) where it conveys a microporous pad that comprises an array of electrodes configured in a bi-polar configuration to ohmically heat dermal tissue beneath the epidermis. Later, Basil Hantash was first to publish clinical results on a novel minimally invasive bipolar microneedle radiofrequency device in 2009 and with this publication a plethora of MNRF devices have received clearance through the FDA.

MNRF devices have been configured in a variety of ways to differentiate themselves in this predominantly saturated market. In terms of the MNRF microneedles, the needles have been placed in different geometric configurations, the number of needles in the array have been different, insulated with various insulation material to control where the RF from the needle is going to dissipate from, no insulation material used to give a non-insulated RF needle affect, angled the needle from the standard 90 degrees, and controlling when the RF energy is going to be outputted at a targeted depth.

In this experimental study of the histologic evaluation of a novel MNRF system, the study aimed to investigate the radiofrequency propagation of an insulated and non-insulated microneedle when initiated in an in vivo porcine model. The bipolar radiofrequency was initiated at a set depth, set RF conduction time, and set frequency amplitudes 1 or 2 MHz.

In Vivo Treatment of a Porcine Model with Bi-Polar Radiofrequency Using Non-Insulated and insulated needles at frequency amplitudes 1 and 2-MHz

All experimental protocols were approved by the Institutional Animal Care and Use Committee (IACUC) and the methods carried out in accordance with the approved guidelines. Two female Yorkshire pig, weighing approximately 50-60 kg (age commensurate with weight) was used for the experiment. Animals were acquired from an approved vendor and were acclimated under standard housing provisions for 1 week prior to the start of the experiments. Clinical observations were recorded once daily during acclimation. All signs of clinical abnormalities were recorded if any. The animal was examined by a veterinarian and deemed fit before utilized for the study.

The MNRF test device and the RF microneedling was performed on the Yorkshire pig. Needling was performed in a grid like fashion. Animal was anesthetized with telazol IM and maintained on isoflurane in oxygen. Animal was positioned in lateral recumbency and then rolled to the contralateral side mid-procedure to utilize available skin surface area. Skin of the thoracic and dorsal regions were shaved and prepped. Grids were marked with indelible ink to denote device application sites. Each application site was spaced 1-cm apart to minimize RF and thermal effects from the effects from the adjacent treatment areas. Device applications continued until usable skin surface area was maximized. A total of 56 applications were performed on the animal.

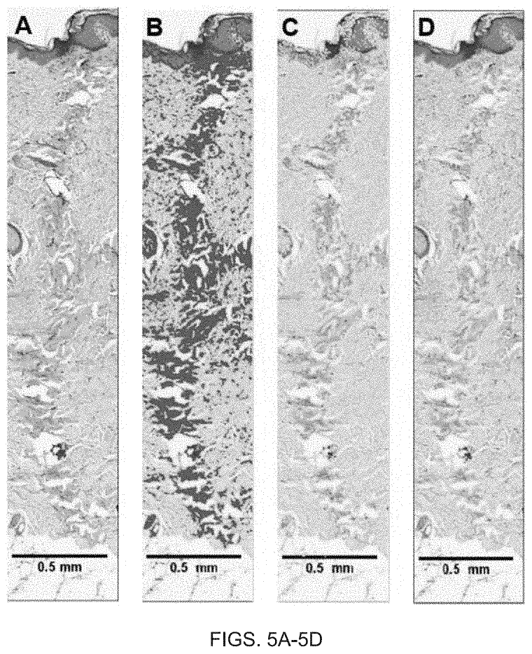

Followed ˜1 hour after each application, a punch biopsy (3-5 mm) was taken of each single needle site. Naïve skin biopsies were also taken for controls. Treated and control skin biopsies (three step sections through each lesion) were fixed in 10% NBF, embedded in paraffin and then stained with Hematoxylin and Eosin (H&E). Samples were then compared for histologic analysis to the naïve tissue biopsies using ImageJ (NIH, 2018) image processing tool. Area analysis was also done on the histologic images to obtain areas of coagulation for comparison purposes for a variety of parameters (FIGS. 5A-5D).

Image Analysis of Electrocoagulation Area after Bi-Polar Radiofrequency In Vivo Treatment

Using ImageJ (NIH, 2018) an area analysis was performed to analyze the area of coagulation after bi-polar radiofrequency in vivo treatment using an insulated and non-insulated microneedle at 1 and 2-MHz. The representative electrocoagulation image was first identified (FIG. 5A) and then using the threshold color the coagulation was isolated using the Hue, Saturation, Brightness (HSB) color space with a red threshold color and the ImageJ default thresholding method (FIG. 5B). Once isolated, the threshold image was selected and an area analysis was then taken for initial area value (FIG. 5C). The selection tool was then used to isolate the coagulation further to obtain a more isolated area value (FIG. 5D). The final area was then compared with the initial area value that was obtained with the raw selection to verify analysis accuracy.

### Thermal Imaging of MNRF Device Treatment of Porcine Skin

All experimental protocols were approved by the Institutional Animal Care and Use Committee (IACUC) and the methods carried out in accordance with the approved guidelines. Three Yucatan Minipigs, weighing approximately 20 kg were required for the thermal imaging analysis study of the novel MNRF system. Animals were acquired and acclimated under standard housing provisions for at approximately 1 week prior to start of study.

Animals were anesthetized and monitored routinely and placed in sternal recumbency. Surgical procedures were performed by a qualified veterinarian. An approximately 10 cm×10 cm dorsal skin flap were created and elevated towards midline. The animal was positioned in lateral recumbency such that the skin flat is retracted parallel to the table. The skin flap thickness was then measured by caliper at the proximal, medial, and distal locations of the flap. The thicknesses were recorded and the depth parameters were then optimized for the experiment. For optimization, the depth of the needles were 1 millimeter away from the bottom of the skin flap. This was due to obtaining optimal thermal imaging viewing and minimizing the chance that the environment around the skin flap effects the heat transfer phenomena.

The thermal imaging system (FLIR A325 sc) was positioned underneath the flap (dermal surface). A MNRF treatment was then performed on the epidermal surface. The started video will be established when the needles penetrate fully through the skin. The video was then exported and filtered using MATLAB (The MathWorks Inc., US) software provided by FLIR, US to obtain a clean image of the heating phenomena produced by the MNRF system.

Additional flaps were then made as needed along the dorsum, utilizing available skin surface. Cautery may have been used on some experimental flaps to achieve hemostasis along the skin flap margins. Veterinary recommendation was also used followed for all procedural details. Animals were then euthanized under general anesthesia at the conclusion of the procedure with an IV overdose of commercially available euthanasia solution.

In Vivo Tissue Reactions after Invasive, Pulsed-Type, Bipolar RF Treatment of Porcine Skin

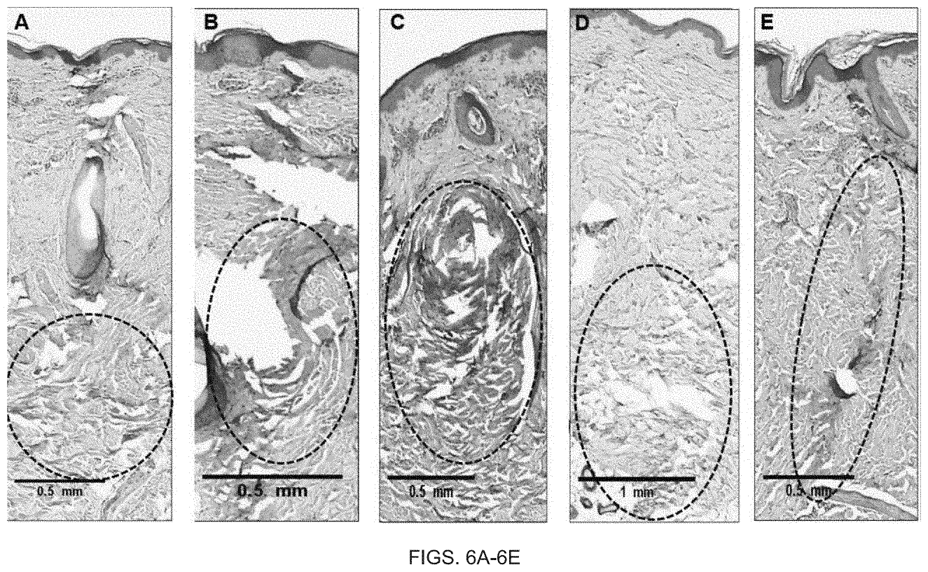

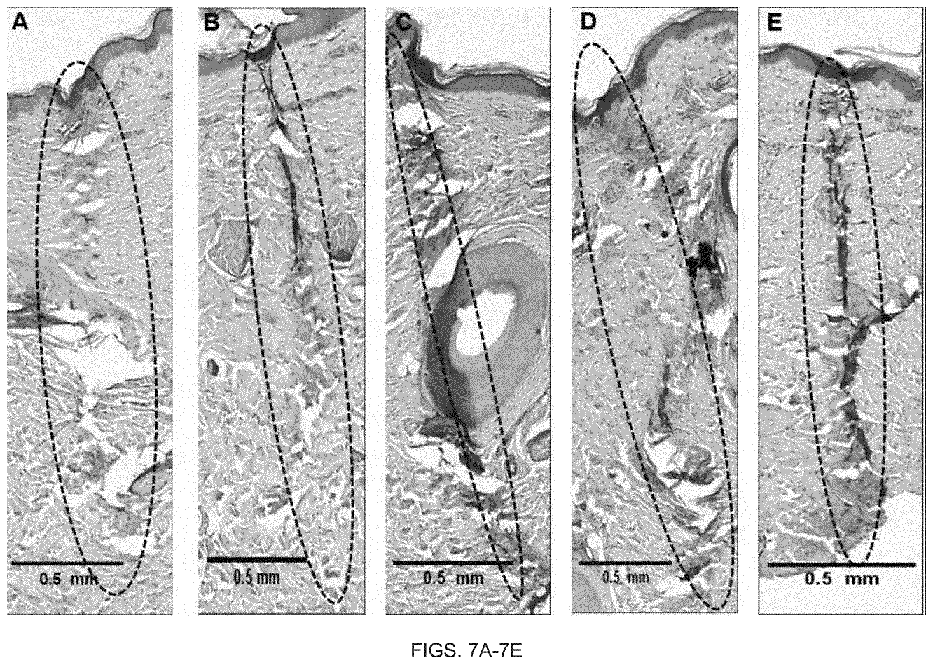

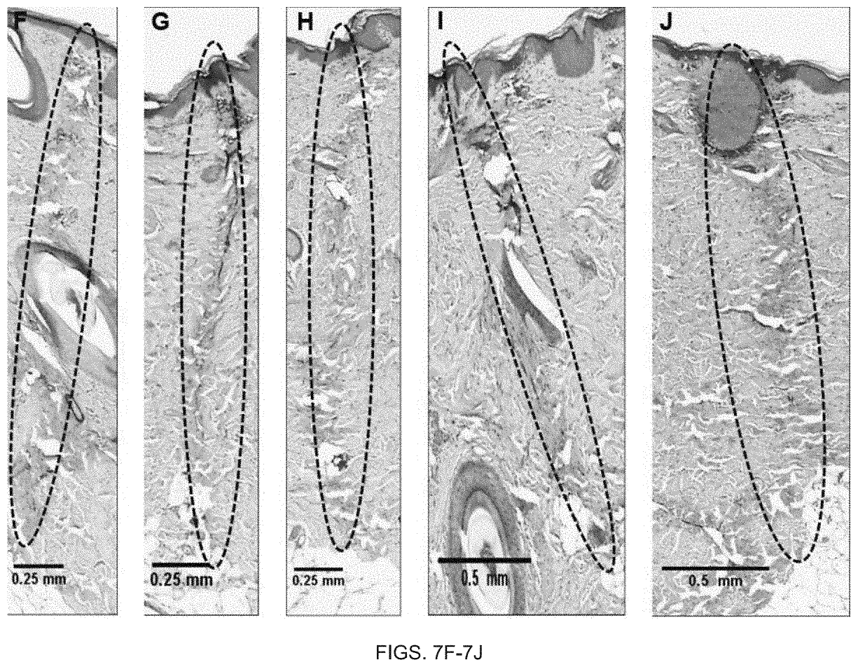

Immediately after RF treatment, skin samples exhibited coagulation of thermal injury where the needle was initiated within the porcine model. In the histologic images (FIGS. 6A-E, FIGS. 7A-J), the epidermis showed where the needle penetrated based off the darker purple section, but did not show significant damage, if any, to the epidermis. For MNRF treatments, it is important to spare the epidermis from thermal damage because with no thermal damage to the epidermis the patient would have little downtime and if treating Fitzpatrick skin type V-VI the user would want to use a modality that avoids the risk of post-inflammatory hyperpigmentation (PIH), transient or permanent hypopigmentation from occurring. Radiofrequency shows to be the modality of choice when avoiding such adverse events.

Area Analysis of In Vivo Tissue Reactions after MNRF Treatment of Porcine Skin

Through area analysis of the thermal injury, one can see a positive correlation for 1 MHz signal amplitude where when power is increased the energy radiates out farther causing a larger coagulation (TABLE 1). For the insulated needles (FIGS. 6A-E, 7A-E), the thermal injury, from a histologic visual, shows the energy at 2 MHz to be more narrow and possibly more concentrated than using 1 MHz signal amplitude. This may be due to the period of the wave being greater at 1 MHz to 2 MHz, allowing a higher voltage and current density gradient to move through the plated microneedles to establish the electrocoagulation effect and radiate for a longer distance. The area of coagulation did not fluctuate as much with higher power level as shown in the 2 MHz insulated needle coagulation areas in TABLE 1, which follows the observations in previous studies where the coagulation was significantly affected with microneedle depth and RF conduction times. Electrocoagulations also have variability due to bioimpedance or the resistance of the targeted tissue when using the RF modality.

Comparing Insulated and Non-Insulated Thermal Injury after MNFR Treatment of Porcine Skin

The technical difference between an insulated needle and a non-insulated needle is that for the insulated needle, the needle is coated with a insulation material where only 0.3-mm of the needle is not insulated and therefore energy can radiate out from the non-insulated spacing. The study showed the tissue reaction for 1 MHz/2 MHz insulated needle and 1 MHz/2 MHz non-insulated needle. 2 MHz non-insulated needle thermal injury was not shown due to lack of verification of coagulation effect when histology analysis took place. The study showed how using an insulated needle at 2 MHz gives a similar histologic coagulation compared to a 1 MHz non-insulated needle (FIGS. 7F-J). This would not follow the general hypothesis of an insulated needle though because one would think that if the needle is insulated then no RF energy would be dispersing from the needle. When comparing 1 MHz insulated (FIGS. 6A-E) to 2 MHz insulated (FIGS. 7A-E), the 1 MHz coagulations have more ovular shape compared to 2 MHz that is more columnar in shape. The reasoning behind why a frequency dispersion may happen at 2 MHz when using a insulated needle may be due to parasitic effects of the material (including series resistance and the back metal contact of the insulation material that may be creating a lossy interfacial layer), the frequency dependence of the dielectric constant (k-value), the relaxation behavior of the insulation material under the applied radiofrequency, and the quantum mechanical effect of the insulation material. This finding opens up a new paradigm of RF microneedling where the user could just toggle between frequencies to establish an insulated or a non-insulated microneedle.

TABLE 1
Area Analysis of Electrocoagulation
(A) 1 MHz Non-Insulated Area (mm²)

| LEVEL 4 | LEVEL 5 | LEVEL 6 | LEVEL 7 | LEVEL 8 |
|---|---|---|---|---|
| 0.149 | 0.109 | 0.304 | 0.206 | 0.451 |

(B) 1 MHz Insulated Area (mm²)

| | | | | |
|---|---|---|---|---|
| LEVEL 4 | LEVEL 5 | LEVEL 6 | LEVEL 7 | LEVEL 8 |
| 0.168 | 0.233 | 0.470 | 0.660 | 0.341 |

(C) 2 MHz Insulated Area (mm²)

| | | | | |
|---|---|---|---|---|
| LEVEL 4 | LEVEL 5 | LEVEL 6 | LEVEL 7 | LEVEL 8 |
| 0.304 | 0.194 | 0.287 | 0.291 | 0.266 |

Thermal Temperature Image Analysis after MNRF Treatment of Porcine Skin.

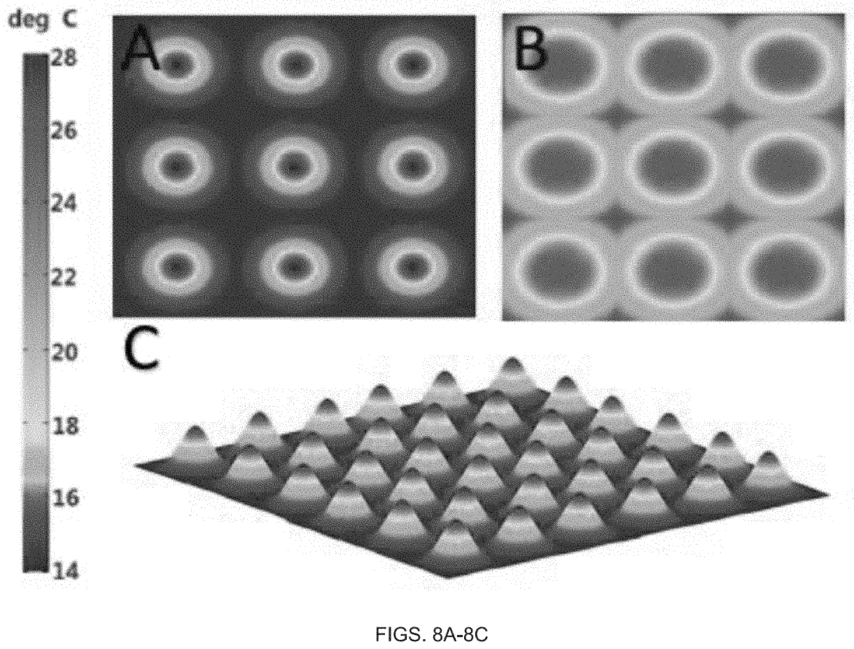

Some of the advantages conferred by the current disclosure include uniform heating. When a MNRF system delivers energy to a specific skin layer, energy comes out of the microneedles (electrodes) producing an electrocoagulation shown previously in FIGS. 6A-6E and 7A-7J. In FIGS. 8A-8C it shows an hypothesis of how the energy dispersion occurs when using an MNRF system under a IR camera in an ideal system.

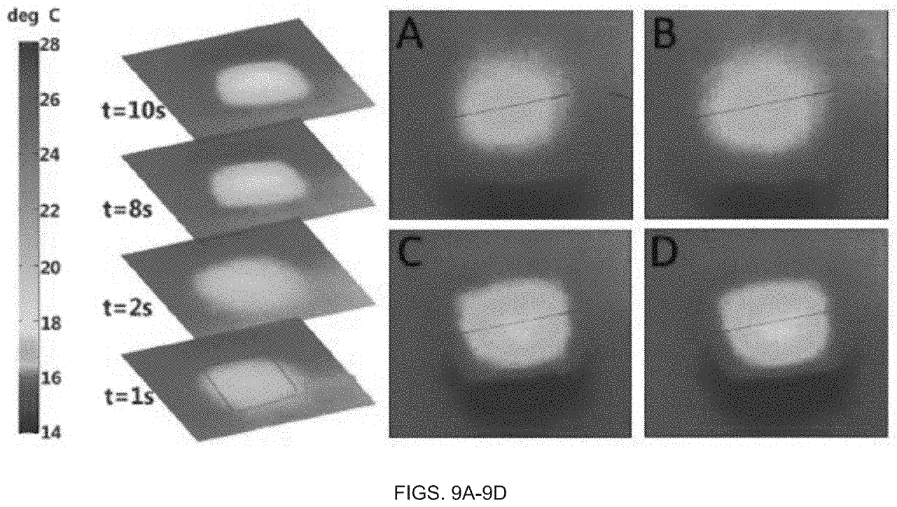

In FIGS. 9A-9D, the IR camera shows a different result—a uniform block of radiofrequency energy dispersion. Also, it shows that even though the max RF conduction time is 800-ms the heating does not fully saturate until ˜10 s. Uniform heating may provide maximum cell stimulation. The uniform heating may also provide cell stimulation and recruitment in the target area or the area of the microneedle array 110. In some embodiments, fractionation or fractional heating does not occur within the dermis. The purpose of the fractionation of energy or fractional photothermolysis (FP) was believed to be safer and more efficacious modality over ablative skin resurfacing (ASR) when dealing with epidermal damage. ASR patients would experience edema, oozing, crusting, and burning discomfort. For FP patients the epidermis repairs faster due to small wounds and short migratory paths for keratinocytes. Due to the use of radiofrequency microneedling (RFMN) being non-ablative dermal remodeling or given the option to the user where they can spare the epidermis from injury, RFMN does not need to necessarily fractionate. Utilizing uniform heating or complete thermal diffusion where the whole area is stimulated. Thus, uniform heating may provide superior results compared to fractional heating.

In some embodiments, the microneedling system 100 may be configured to deliver uniform heating. In this configuration, the uniform heating may occur with enhanced cell stimulation. In some instances, frequencies of the device can be toggled where the electrical impedance of the needle insulation is much lower than the frequency selected and therefore the needle would exhibit the properties of non-insulation or approaching the properties of non-insulation. In some embodiments, the semi-insulated properties due to the insulation may result in semi-insulating at some frequencies. In some embodiments, the insulation can be throughout the whole microneedle 112. In some embodiments, the insulation is selectively coated on the needle. In some instances, the coating can be made to modulate power through a needle shaft to selectively heat a microneedle 112. In some instances, the microneedle 112 is uniformly heated. In some instances, the microneedle 112 is uniformly heated while taking into account the bioimpedance of the skin layers the microneedle is penetrating. This may be an advantageous because a non-insulated needle may heat one skin layer more than the other due to the differences in bioimpedance between the layers.

In some embodiments, the uniform heating may produce a fractional pattern that does not cause fractional damage. In some embodiments, the enhanced cell stimulation may be a result of each island having a separate channel for RF energy where the bioimpedance change is regulated for uniform heating or thermal response to occur. In some embodiments, the skin's bioimpedance and non-conduct area protect against adverse events. In some embodiments, the adverse events may be selected from edema, oozing, crusting, blistering, burning, discomfort, and scarring. In some instances, the whole area is to some extent damaged. Further, with the use of microneedles 112 the user can control where the energy is transmitted versus a laser where the energy can disperse based on the environment and the user cannot control the depth of heating without increasing the risk of adverse events that are produced from severe epidermal damage. With RFMN, you can establish uniform heating at any depths based off the adjustments of the microneedle placement.

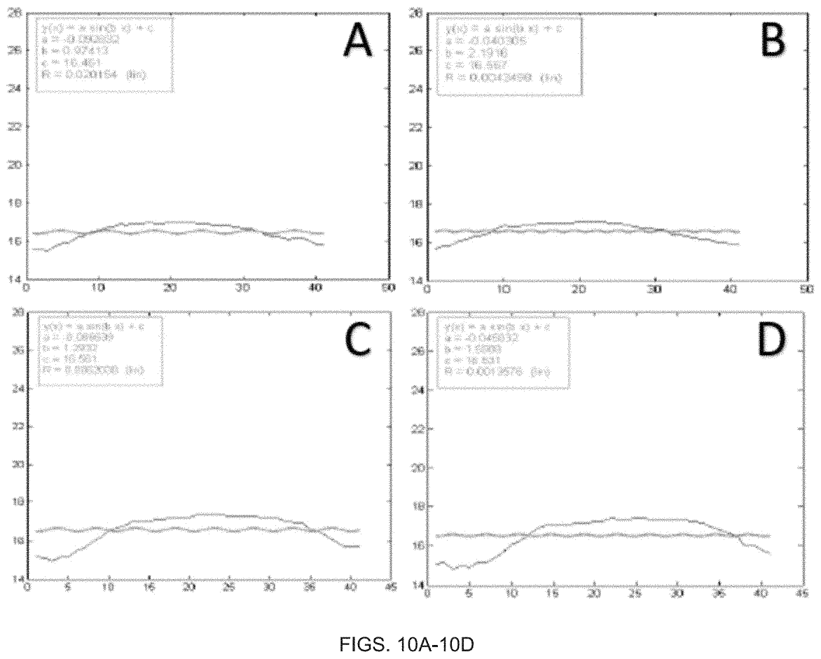

Next, when comparing the thermal profile to a sinusoidal wave the result was there was no evidence of periodic heating in the IR images of heated tissue that were 1 mm deep from the electrodes of the MNRF system (FIGS. 10A-10D). At best, less than 2% of the data showed a repeating pattern and therefore FIGS. 8A-8C's hypothesis cannot be supported.

Heat transfer occurs when there is a physical act of thermal energy being exchanged between two systems by dissipating heat. Therefore, heat cannot be just described as temperature, but it must also include the flow of heat. The amount of thermal energy is determined by the temperature and heat flow represents movement of the movement of the thermal energy. Heat transfer can be grouped into three categories: conduction, convection, and radiation. All three heat transfer methods happen when applying a MNRF system to a specific skin layer. At high level, conduction occurs between the heated needle and the tissue, convection occurs between the heated needle and the blood and other fluids in the skin, and radiation happens with the radiofrequency dispersion where no medium is needed. All three of these heat transfer methods used for the tissue can have resulted in the block heating versus the periodic, non-uniform heating that FIGS. 8A-8C hypothesized. FIGS. 8A-8C just take into account the conduction between the needles and the tissue, when in experiment the system is much more complex.

Then if there is block heating or uniform heating occurring, why is there coagulations appearing in FIGS. 6A-6E and 7A-7J? Not to be bound by a particular theory, this may be due to another heating phenomena called the Seebeck effect. The Seebeck effect is a phenomenon in which a temperature difference between two dissimilar electrical conductions produce a voltage difference between the two substances. In the experiments case, this could be positive electrode to negative electrode or it could be positive or negative electrode to skin tissue. This voltage difference may have a larger effect than the dispersion of heat and may result in the coagulations that are seen in FIGS. 6A-6E and 7A-7J, while the MNRF system still produces a block heating or uniform heating effect. Another phenomena to investigate would be the spin Seebeck effect where when taking account a polarizable material in a thermal gradient, a spin voltage and current can be created proportional to the temperature difference that could be the cause of the electrocoagulation when using the MNRF device.

### Conclusion

The experimental study outlined the structural changes when bipolar radiofrequency was initiated inside a in vivo porcine skin model using an insulated and non-insulated needle at signal amplitudes of 1 MHz and 2 MHz. The findings also suggest that using 2 MHz insulated needle can induce similar histologic change compared to 1 MHz non-insulated needle, where this is the first MNRF device to observe and show this. Further experiments are being established to further verify the insulation and non-insulation effect when toggling between the two frequencies of the novel MNRF device. The findings also suggest that an MNRF system may produce a uniform heating block and also still produce an electrocoagulation due to certain heating phenomena. Further experiments and observations with a larger sample size have to occur to further validate the novel MNRF device described in this paper and although the in vivo porcine skin model is the closest to a human skin model, it may not directly coincide and therefore more studies have to be done through a human clinical trial using the MNRF device described.

Accordingly, some aspects described herein relate to the following numbered alternative:

1. A device for delivering electrical energy to the skin of a patient, the device comprising: an array comprising a plurality of electrodes arranged across the array, each electrode being configured to be placed into contact with the skin of the patient and/or to be inserted into the skin of the patient, wherein the plurality of electrodes comprises a plurality of positive electrodes and a plurality of negative electrodes, wherein each negative electrode is positioned adjacent to a positive electrode, and wherein each positive electrode is positioned adjacent to a negative electrode.

2. The device of alternative Error! Reference source not found., wherein the array comprises alternating rows or columns of positive and negative electrodes.

3. The device of alternative 1, wherein the plurality of electrodes comprise a plurality of central electrodes, each central electrode being uniformly surrounded by at least three adjacent closest electrodes of an opposite polarity.

4. The device of alternative 3, wherein each central electrode is uniformly surrounded by at least four adjacent closest electrodes of an opposite polarity.

5. The device of alternative 4, wherein the plurality of negative electrodes and the plurality of positive electrodes are arranged in a checkerboard fashion.

6. The device of alternative 4, wherein each central electrode is uniformly surrounded by at least five adjacent closest electrodes of an opposite polarity.

7. The device of alternative 6, wherein the plurality of electrodes are arranged in a pentagonal pattern.

8. The device of alternative 6, wherein each central electrode is uniformly surrounded by at least six adjacent closest electrodes of an opposite polarity.

9. The device of alternative 8, wherein the plurality of electrodes are arranged in a hexagonal pattern.

10. The device of any of the preceding alternatives, wherein the array forms at least one zone bordered by at least three electrodes such that neither a path between any one of the plurality of positive electrodes and a closest negative electrode nor a path between any one of the plurality of negative electrodes and a closest positive electrode crosses the zone.

11. The device of any one of the preceding alternatives, wherein the plurality of electrodes comprises a 1:1 ratio of negative electrodes to positive electrodes.

12. The device of any one of alternatives 1 to 10, wherein the plurality of electrodes comprises more than a 1:1 ratio of negative electrodes to positive electrodes.

13. The device of any one of alternatives 1 to 10, wherein the plurality of electrodes comprises less than a 1:1 ratio of negative electrodes to positive electrodes.

14. The device of any one of alternatives 3 to 13, wherein at least one central electrode shares a closest adjacent electrode of opposite polarity with another central electrode.

15. The device of any one of alternatives 3 to 13, wherein none of the central electrodes shares a closest adjacent electrode of opposite polarity with another central electrode.

16. The device of any one of alternatives 3 to 15, wherein each central electrode is a plate surface electrode.

17. The device of alternative 16, wherein the plate surface electrodes are polygonal.

18. The device of alternative 17, wherein the polygon has the same number of sides as the number of adjacent closest electrodes of opposite polarity.

19. The device of any one of alternatives 3 to 18, wherein all of the adjacent closest electrodes of opposite polarity are microneedles.

20. The device of any one of alternatives 16 to 19, wherein the surface plate electrode has a cross-sectional area at least 1.5× larger than the cross-sectional area of all of the adjacent closest electrodes of opposite polarity.

21. The device of any one of the preceding alternatives, wherein the plurality of electrodes are distributed between a plurality of islands such that all of the electrodes inside an island are closer to an electrode of opposite polarity inside the island than an electrode of opposite polarity outside the island and such that all the electrodes outside an island are closer to an electrode of opposite polarity outside the island than an electrode of opposite polarity inside the island.

22. The device of alternative 21, wherein all of the electrodes outside any one of the islands are positioned further from electrodes inside the island than any electrode inside the island is positioned relative to another electrode inside the island.

23. The device of alternative 21, wherein at least one electrode outside of each of the islands is positioned as close to an electrode inside the island as the electrode inside the island is positioned to another electrode inside the island.

24. The device of any one of the preceding alternatives, wherein each electrode from the plurality of electrodes is positioned substantially the same distance from a closest electrode of opposite polarity.

25. The device of any one of the preceding alternatives, wherein all electrodes of a first polarity are microneedles and wherein all electrodes of a second polarity are surface electrodes.

26. The device of alternative 25, wherein all of the positive electrodes are microneedles and all of the negative electrodes are surface electrodes.

27. The device of any one of the preceding alternatives, wherein either the positive electrodes or the negative electrodes are connected to electrical ground.

28. The device of any one of the preceding alternatives, further comprising a handpiece, wherein the array is positioned on a distal end of the handpiece.

29. The device of alternative 28, wherein the array comprises at least one microneedle configured to be inserted into the skin of the patient, and wherein the handpiece is configured to reciprocate the array such that the at least one microneedle punctures the skin of the patient.

30. The device of alternative 29, wherein the device is configured to deliver the electrical energy during a period coinciding with a full insertion of the at least one microneedle into the skin of the patient.

31. The device of any one of the preceding alternatives, wherein the device is configured to deliver the electrical energy to the skin of the patient between the plurality of positive electrodes and the plurality of negative electrodes in a waveform having frequency within the radiofrequency range.

32. The device of any one of the preceding alternatives, wherein the device is configured to raise the temperature of the skin to a degree sufficient to induce collagen coagulation.

33. The device of alternative 32, wherein the device is configured to leave a plurality of areas of skin encompassed by the array during delivery of the electrical energy, effectively untreated such that collagen is not coagulated within these areas.

34. The device of any one of the preceding alternatives, wherein the array comprises at least one microneedle configured to penetrate the skin of the patient such that a distal tip of the needle reaches the dermis.

35. A device for delivering radio frequency energy to a dermis of a patient, the device comprising: at least one microneedle configured to penetrate the skin of the patient such that a distal tip of the microneedle reaches the dermis, wherein the microneedle comprises at least one substrate enclosed by one material, wherein the microneedle is configured to deliver a dispersion of radio frequency energy into the dermis, whereby tissue of the dermis is damaged, and wherein the microneedle is further configured to exhibit either a conducting property or a nonconducting property.

36. The device of alternative 35, wherein the at least one microneedle is configured to penetrate the subcutaneous layer.

37. The device of alternative 35, wherein the at least one microneedle is configured to penetrate the epidermis, dermis, and hypodermis.

38. The device of any one of alternatives 35 to 37, wherein the at least one microneedle is inserted no more than 6 mm into the skin.

39. The device of any one of alternatives 35 to 37, wherein the bioimpedance of the skin and non-conduct area protect against adverse events.

40. A device for delivering radio frequency energy to a dermis of a patient, the device comprising: at least one contact plate configured to be in contact with the skin of the patient, wherein the contact plate comprises at least one substrate enclosed by one material, wherein the contact plate is configured to deliver a dispersion of radio frequency energy into the dermis, whereby tissue of the dermis is damaged, and wherein the contact plate is further configured to exhibit either a conducting property or a nonconducting property.

41. The device of alternative 38, wherein the contact plate is configured to substantially exhibit either a conducting property or a nonconducting property based off the bioimpedance of the skin.

42. The device of any one of alternatives 35 to 41, further comprising a processor configured to adjust a parameter of the radio frequency energy to select between the conducting property and the nonconducting property, wherein the parameter is at least one member selected from the group consisting of frequency, current, voltage, and conduction time.

43. The device of alternative 42, wherein the material has a preselected dielectric relaxation value corresponding to the parameter.

44. The device of any one of alternatives 35 to 43, wherein a thickness of the material is selected such that the conductive properties of the microneedle are preselected.

45. The device of alternative 44, wherein the material has a preselected dielectric relaxation value corresponding to thickness of the material.

46. The device of any one of alternatives 35 to 45, wherein the radiofrequency energy is microwave energy.

47. The device of any one of alternatives 35 to 46, wherein damaging comprises induction of collagen coagulation.

48. The device of any one of alternatives 35 to 47, wherein the material is an amorphous material.

49. The device of any one of alternatives 35 to 48, wherein the material is a dielectric thin film.

50. The device of any one of alternatives 35 to 49, wherein the material is an oxide, optionally one or more of a silicon oxide, a zirconium oxide, a lanthanum oxide, a lanthanum zirconium oxide or a cerium oxide.

51. The device of any one of alternatives 35 to 49, wherein the material is a polymeric material, optionally an unsubstituted paracyclophane, a substituted paracyclophane, dichloro-di-p-xylylene, dichloro-paracyclophane, or trichloro-paracyclophane.

52. The device of any one of alternatives 35 to 49, wherein the material is a metal oxide semiconductor.

53. The device of any one of alternatives 35 to 52, wherein the material has a high thermal conductivity.

54. The device of any one of alternatives 35 to 53, wherein the substrate is a metal or a metal alloy, optionally iron, titanium, nickel, aluminum, or chromium, steel, or stainless steel.

55. The device of alternative 40, wherein the radio frequency energy is laser energy, and wherein the contact plate is configured to transition the laser energy from a fraction pattern to a nonfractional pattern.

56. The device of alternative 55, wherein the fractional pattern is provided by an array of apertures.

57. The device of alternative 56, wherein the apertures are selected from the group consisting of holes and slots.

58. The device of alternative 55, wherein the fractional pattern is provided by a patterned array of emitters of laser energy.

59. The device of any one of alternatives 55 to 58, wherein the fractional pattern is regular.

60. The device of any one of alternatives 55 to 58, wherein the fractional pattern is irregular.

61. A skin treatment method, comprising: inserting a plurality of needles into a layer of skin; and regulating delivery of a radiofrequency (RF) energy form a RF energy source to the plurality of needles to induce a substantially uniform area of heating by the RF energy in a dermal layer.

62. The method of alternative 61, wherein the RF energy source to the plurality of needles does not induce a fractional area of heating by the RF energy in a dermal layer.

63. The method of alternatives 61 to 62, wherein the substantially uniform area of heating produces fractionated coagulation.

64. The method of alternatives 61 to 63, wherein the substantially uniform area of heating produces a biological stress response in the dermal layer.

65. The method of alternative 64, wherein the biological stress response is caused by a bioimpedance change.

66. The method of alternative 65, wherein the biological stress response caused by a bioimpedance change is a cell swelling, cell stimulation, or cellular bystander effect.

67. The method of alternatives 61 to 66, wherein the plurality of needles is attached to a handpiece.

68. The method of any one of alternatives 61 or 67, wherein the plurality of needles is configured to receive RF energy from the RF energy source.

69. The method of any one of alternatives 61 to 68, wherein regulating delivery of the RF energy is configured to stimulate formation of new collagen in the target skin layer.

70. The method of any one of alternatives 62 to 69, wherein regulating delivery of the RF energy includes controlling a RF waveform.

71. The method of any one of alternatives 62 to 70, wherein regulating delivery of the RF energy includes controlling power transmitted from the plurality of needles.

72. The method of any one of alternatives 62 to 71, wherein the needle-to-needle width of the plurality of needles is between about 0.1 mm and 0.5 mm.

73. The method of any one of alternatives 62 to 72, wherein regulating delivery of the RF energy includes RF conduction time.

74. The method of any one of alternatives 62 to 73, wherein regulating delivery of RF energy further includes a bioimpedance change in the layer of skin.

75. The method of alternative 74, wherein the bioimpedance change is a thermal response from the method of alternative 61. The method of alternative 74, wherein a thermal response is throughout the area of the microneedle area.

76. The method of any one of alternatives 62 to 75, wherein regulating delivery of RF energy further includes a substantially uniform thermal profile encompassed by the plurality of needles.

77. The method of alternative 76, wherein the substantially uniform thermal profile encompasses the plurality of needles xy boundaries.

78. The method of alternative 77, wherein the thermal profile indicate a bioimpedance change through the target skin layer.

79. The method of alternative 78, wherein the bioimpedance indicates an electrophysiological change is a biological stress response, cell stimulation, cell swelling, cellular bystander effect, cell damage, heat shock response, a coagulating effect, a necrosis of the treated skin, or a denaturation effect.

80. The method of any one of alternatives 65 to 79, wherein regulating the delivery of RF to adjust the RF waveform to be substantially equivalent to the input waveform and output waveform.

81. The method of any one of alternatives 69 to 80, wherein regulating the delivery of RF is to adjust the RF conduction time.

82. The method of any one of alternatives 68 to 81, wherein regulating the delivery of RF is to adjust the power applied to the layer of skin.

83. The method of any one of alternatives 70 to 82, wherein regulating the delivery to RF adjusts the power applied as the bioimpedance of the layer of skin changes.

84. The method of any one of alternatives 65 to 82, wherein the uniform area of heating by the RF energy is sufficient for a biological stress response, cell stimulation, cell swelling, cellular bystander effect, cell damage, heat shock response, a coagulating effect, a necrosis of the treated skin, or a denaturation effect within the target skin layer.

85. The method of any one of alternatives 70 to 84, wherein regulating the delivery of RF causes thermal diffusion that continues for longer than the set RF conduction time.

86. The method of any one of alternatives 65 to 85, wherein the substantially uniform area is the sum of thermal application and the thermal dispersion after RF conduction time.

87. The method of any one of alternatives 65 to 86, wherein the plurality of needles are spaced out with a width of no more than 0.5 mm.

88. The method of any one of alternatives 65 to 87, wherein the uniform area of heating is within the horizontal plane of the layer of skin and a treatment area.

89. The method of any one of alternatives 65 to 88, wherein the uniform area of heating is within the vertical plane of the target skin layer and treatment area.

90. The method of any one of alternatives 65 to 89, wherein the uniform area of heating vertical plane is controlled by the height of a non-insulated area of the needle.

91. The method of any one of alternatives 65 to 90, wherein the microneedles are configured as monopolar electrodes.

92. The method of any one of alternatives 65 to 91, wherein the microneedles are configured as bipolar electrodes.

93. The method of any one of alternatives 65 to 92, wherein the microneedles are configured as unipolar electrodes.

94. A skin treatment method, comprising: inserting a plurality of needles into a layer of skin; and regulating delivery of the RF energy from the RF energy source to the plurality of needles to induce a substantially uniform area of heating by the RF energy in the skin when the needles are inserted therein, wherein an ultrasonic element adjacent to the at least two needles verifies the uniform heating for optimized feedback.

95. The method of alternative 94, wherein the skin may include the epidermal layer, the dermal layer, and the hypodermal layer.

96. The method of alternative 94, wherein the ultrasonic element are used for imaging of the thermal dispersion.

97. The method of any one of alternatives 94 to 96, wherein the ultrasonic element produces high intensity focused ultrasound.

98. The method of alternative 97, wherein the high intensity focused ultrasound converges with the RF for a treatment modality.

99. The method of any one of alternatives 94 to 98, wherein the ultrasonic element is an ultrasonic transducer.

100. The method of any one of alternatives 94 to 99, wherein the ultrasonic element is an acoustic transducer.

101. The method of any one of alternatives 94 to 100, wherein the ultrasonic element produce acoustic waves to treat the target skin layer with Acoustic Wave Therapy (AWT).

102. The method of alternative 94, wherein the AWT and RF energy treats cellulite.

103. The method of any one of alternatives 94 to 101, wherein the acoustic waves vibrates at least one needle.

104. The method of any one of alternatives 94 to 103, wherein the needles do not vibrate.

105. The method of any one of alternatives 94 to 104, wherein regulating delivery of the RF energy includes controlling the RF waveform, needle-to-needle width, and RF conduction time.

Publications related to radiofrequency microneedle treatment include the following: Cho, S. I., Chung, B. Y., Choi, M. G., Baek, J. H., Cho, H. J., Park, C. W., . . . & Kim, H. O. (2012). Evaluation of the clinical efficacy of fractional radiofrequency microneedle treatment in acne scars and large facial pores. Dermatologic Surgery, 38(7pt1), 1017-1024; Peterson, J. D., Palm, M. D., Kiripolsky, M. G., Guiha, I. C., & Goldman, M. P. (2011). Evaluation of the effect of fractional laser with radiofrequency and fractionated radiofrequency on the improvement of acne scars. Dermatologic Surgery, 37(9), 1260-1267; Man, J., & Goldberg, D. J. (2012). Safety and efficacy of fractional bipolar radiofrequency treatment in Fitzpatrick skin types V-VI. Journal of Cosmetic and Laser Therapy, 87(6), 179-183; Lee, S. J., Goo, J. W., Shin, J., Chung, W. S., Kang, J. M., Kim, Y. K., & Cho, S. B. (2012). Use of Fractionated Microneedle Radiofrequency for the Treatment of Inflammatory Acne Vulgaris in 18 Korean Patients. Dermatologic Surgery, 38(3), 400-405; Utely, D. (1999). U.S. Pat. No. 6,277,116B1. Washington, D.C.: U.S. Patent and Trademark Office; Hantash, B. M., Renton, B., Berkowitz, R. L., Stridde, B. C., & Newman, J. (2009). Pilot clinical study of a novel minimally invasive bipolar microneedle radiofrequency device. Lasers in Surgery and Medicine: The Official Journal of the American Society for Laser Medicine and Surgery, 41(2), 87-95; Zheng, Z., Goo, B., Kim, D. Y., Kang, J. S., & Cho, S. B. (2014). Histometric analysis of skin-radiofrequency interaction using a fractionated microneedle delivery system. Dermatologic Surgery, 40(2), 134-141; Van, N. B., & Alster, T. S. (2009). Laser treatment of dark skin: a review and update. Journal of drugs in dermatology: JDD, 8(9), 821-827; Man, J., & Goldberg, D. J. (2012). Safety and efficacy of fractional bipolar radiofrequency treatment in Fitzpatrick skin types V-VI. Journal of Cosmetic and Laser Therapy, 87(6), 179-183; Taheri, A., Mansoori, P., Sandoval, L. F., Feldman, S. R., Pearce, D., & Williford, P. M. (2014). Electrosurgery: part II. Technology, applications, and safety of electrosurgical devices. Journal of the American Academy of Dermatology, 70(4), 607-el; Brill, A. I. (2008). Bipolar electrosurgery: convention and innovation. Clinical obstetrics and gynecology, 51(1), 153-158; Taheri, A., Mansoori, P., Sandoval, L. F., Feldman, S. R., Pearce, D., & Williford, P. M. (2014). Electrosurgery: part I. Basics and principles. Journal of the American Academy of Dermatology, 70(4), 591-el; Tao, J., Zhao, C. Z., Zhao, C., Taechakumput, P., Werner, M., Taylor, S. and Chalker, P. R., 2012. Extrinsic and intrinsic frequency dispersion of high-k materials in capacitance-voltage measurements. Materials, 5(6), pp. 1005-1032; Uchida, K., Takahashi, S., Harii, K., Ieda, J., Koshibae, W., Ando, K., Maekawa, S. and Saitoh, E., 2008. Observation of the spin Seebeck effect. Nature, 455(7214), p. 778.

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Where a range of values is provided, it is understood that the upper and lower limit, and each intervening value between the upper and lower limit of the range is encompassed within the embodiments. All numbers expressing, e.g., operating conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

In addition, where features or aspects of the disclosure are described in terms of Markush groups, those skilled in the art will recognize that the disclosure is also thereby described in terms of any individual member or subgroup of members of the Markush group.

What is claimed is:

1. A skin treatment method, comprising:
inserting a plurality of needles into a layer of skin; and
regulating delivery of a radiofrequency (RF) energy form a RF energy source to the plurality of needles to induce a substantially uniform area of heating by the RF energy in a dermal layer,
wherein the RF energy source to the plurality of needles does not induce a fractional area of heating by the RF energy in a dermal layer, or wherein the substantially uniform area of heating produces fractionated coagulation, or wherein the substantially uniform area of heating produces a biological stress response in the dermal, epidermal, and hypodermal layer, or wherein the biological stress response is caused by a bioimpedance change, or wherein regulating delivery of RF energy further includes a bioimpedance change in the layer of skin, or wherein regulating the delivery to RF adjusts the power applied as the bioimpedance of the layer of skin changes.

2. A device for delivering radio frequency energy to a dermis of a patient, the device comprising:
at least one microneedle configured to penetrate the skin of the patient such that a distal tip of the microneedle reaches the dermis, wherein the microneedle comprises at least one substrate enclosed by one material, wherein the microneedle is configured to deliver a dispersion of radio frequency energy into the dermis, whereby tissue of the dermis is damaged, and wherein the microneedle is further configured to exhibit either a conducting property or a nonconducting property, wherein the at least one microneedle is configured to penetrate the epidermis, dermis, and hypodermis.

3. A device for delivering radio frequency energy to a dermis of a patient, the device comprising:
at least one contact plate configured to be in contact with the skin of the patient, wherein the contact plate comprises at least one substrate enclosed by at least one material, wherein the contact plate is configured to deliver a dispersion of radio frequency energy into the dermis, whereby tissue of the dermis is damaged, and wherein the contact plate is further configured to exhibit either a conducting property or a nonconducting property; and a processor configured to adjust a parameter of the radio frequency energy to select between the conducting property and the nonconducting property, wherein the parameter is at least one member selected from the group consisting of frequency, current, voltage, and conduction time.

4. A skin treatment method, comprising:
inserting a plurality of needles into a layer of skin; and
regulating delivery of the RF energy from the RF energy source to the plurality of needles to induce a substantially uniform area of heating by the RF energy in the skin when the needles are inserted therein, wherein an ultrasonic element adjacent to the at least two needles verifies the uniform heating for optimized feedback, wherein the skin may include the epidermal layer, the dermal layer, and the hypodermal layer.
